(19) United States
(12) Patent Application Publication
(10) Pub. No.: US 2018/0217125 A1
(43) Pub. Date: Aug. 2, 2018

(54) MEANS AND METHODS FOR THE DETERMINATION OF THE QUALITY OF BLOOD SAMPLES BASED ON A METABOLITE PANEL

(71) Applicant: Metanomics Health, Berlin (DE)

(72) Inventors: Beate Kamlage, Berlin (DE); Oliver Schmitz, Varziner Str.13/14 (DE)

(21) Appl. No.: 15/128,523

(22) PCT Filed: Mar. 26, 2015
(86) PCT No.: PCT/IB2015/052243

§ 371 Date: Sep. 23, 2016

(30) Foreign Application Priority Data
Mar. 26, 2014 (EP) 14161766.2

(51) Int. Cl. G01N 33/49; G06F 19/28; G16H 10/40

(52) U.S. Cl. CPC G01N 33/492; G06F 19/28; G01N 2560/00; G01N 2570/00; G16H 10/40

(57) ABSTRACT

A method for assessing the quality of a blood product sample, comprises determining in said sample the value of the markers of at least one marker panel of the invention, comparing the values determined with corresponding references, and assessing the quality of said blood sample.

The present invention relates to a method for assessing the quality of a blood product sample, comprising determining in said sample the values of the markers of at least one marker panel of the invention, comparing the values determined with corresponding references, and assessing the quality of said blood product sample. The present invention further relates to a device and a kit for assessing the quality of a blood product sample and to a data collection comprising characteristic values of at least the markers of at least one marker panel of the invention and to a data storage medium comprising said data collection. Moreover, the present invention relates to a method of providing a collection of blood products of sufficient quality comprising the steps of the method for assessing the quality of a blood product sample.

The fact that the value of biological material stored in biobanks for any biomedical research or for therapeutic and/or diagnostic purposes may be diminished by pre-analytical confounding factors that interfere with sample composition has been well documented (Aguilar-Mahecha et al. (2012), PLoS ONE 7(6): e38290; Ahmed, F E (2011), Analytical Methods 3: 1029; Beechler et al. (2004), Genes and Immunity 5: 3473); Becker & Lockwood (2013), Clinical Biochemistry 46: 861; Messaoudi et al. (2013), Clinica Chimica Acta 424: 222; Greystoke et al. (2008), Annals of Oncology 19: 990; Hebels et al. (2013), Environmental Health Perspectives 121(4): 480; Kamlage et al. (2014); Clinical Chemistry 60:2: 399; Odozze et al. (2012), Clinical Biochemistry 45: 464; Rai & Vitzthum (2006), Expert Rev Proteomics 3(4): 409; Tuck et al. (2009) J Proteome Res. 8(1): 113; Vaught et al. (2011), J Natl Cancer Inst Monogr 42: 1; Yang et al. Anal. Chem. 85, 2606; Yin et al. (2013), Clinical Chemistry 59(5): 833). E.g., in samples used in metabolite profiling, the potential of biomarker identification and validation may be diminished by pre-analytical confounding factors that interfere with the sample metabolome and may lead to unbalanced systematic bias, increased variability, erratic effects and irreproducible results. Thus, it is decisive to assess the quality of biological material in order to assure quality and suitability for metabolite profiling or other analytical or diagnostic methods. Specifically, confounding factors of relevance are increased time and temperature of blood, plasma or serum sample processing and storage, effects of centrifugation protocol, freezing protocol, and other pre-analytical steps.

There are various standards for quality assurance and quality control for biobanking, e.g., ISO 9001, ISO guide 34, ISO 17025 and others (see, e.g., Carter 2011, Biopreservation and Biobanking 9(2): 157-163; Elliott 2008, Int J Epidemiology 37: 234-244). In order to assess the quality of biological material, at present, biochemical standard parameters, such as nucleic acid content and integrity, presence of coagulation activity, or cellular composition, cell integrity and number of cells in the sample are determined. The evaluation of such standard parameters, however, will not be suitable for all uses of a sample, and may be cost-intensive.

There are reports of protein biomarkers assuring quality of samples for proteome analysis (see, e.g., WO2012/170669). Moreover, it was reported that incubation has an impact on the metabolomic composition of plasma and serum samples (Liu et al. 2010, Anal Biochem 406: 105-115; Fliniaux et al. 2011, Journal of Biomolecular NMR 51(4): 457-465; Boyanton 2002, Clinical Chemistry 48(12): 2242-2247; Bernini et al. 2011, Journal of Biomolecular NMR 49: 231-243).

However, standards for assessing the quality of biological material are not yet available but nevertheless highly desired.

The technical problem underlying the present invention can be seen as the provision of means and methods for complying with the aforementioned needs. The technical problem is solved by the embodiments characterized in the claims and herein below.

Accordingly, the present invention relates to a method for assessing the quality of a blood product sample, comprising:
a) determining in said sample the values of the markers of at least one panel of Table 1;
b) comparing the values determined in step a) with corresponding references, and,
c) assessing the quality of said blood product sample.

The method of the present invention, preferably, is an in vitro method; accordingly, the method, preferably, is a method carried out ex vivo, i.e. not practiced on the human or animal body. Moreover, the method of the present invention may comprise steps in addition to those explicitly mentioned above. For example, further steps may relate, e.g., to calculating an intra-sample ratio for two or more biomarkers for step a), or obtaining additional indicators of quality before or in step c). The method of the present invention is, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches. Preferably, the method for assessing the quality of a blood product sample further comprises determining an external and/or an internal standard for at least one, preferably for all of the markers of said at least one panel. The terms “external standard” and “internal standard” are known to the skilled person.

In preferred embodiments, the method of the present invention additionally comprises one or more of the following steps: i) contacting said blood product sample with an agent specifically interacting with at least one biomarker of the present invention, and determining the amount of a complex formed between said biomarker and said agent specifically interacting with said biomarker; ii) contacting said blood product sample with an enzyme specifically reacting with said at least one biomarker of the present invention, and determining the amount of product formed from said biomarker by said enzyme; iii) contacting said blood product sample with an agent modifying the chemical structure of at least one biomarker, preferably, to form a non-naturally occurring derivative of said biomarker, and detecting said derivative; iv) discarding said blood product sample in case insufficient quality is assessed, and v) excluding said blood product sample from further use in case insufficient quality is assessed.

As used herein, the term “quality” relates to the property of a sample of the present invention of being usable for an intended use, or not. Intended uses for a sample of the present invention are known to the skilled person and include any diagnostic, therapeutic, non-diagnostic, or non-therapeutic use of a sample. Preferably, the intended use is a non-therapeutic use; more preferably, the intended use is a non-therapeutic and non-diagnostic use. Preferably, the intended use is an in vitro use. More preferably, the intended use is an analytic and/or experimental use. Preferred analytic and/or experimental uses of a sample of the present invention are use in genomic analysis, in transcriptomic analysis, or in proteomic analysis. More preferred analytic and/or experimental uses of a sample of the present invention are use in foodomic analysis or in lipidomic analysis. Most preferred analytic and/or experimental uses of a sample of the present invention are use in metabolomics and/or in determining at least one clinically relevant parameter, preferably including clinical chemistry, pharmacokinetic studies, pharmacodynamic studies, and/or molecular diagnostics. In a preferred embodiment, the intended use is use in proteomic analysis.

Accordingly, the term “sufficient quality” relates to the property of a sample of the present invention of providing measurement values unperturbed by sample processing, i.e., preferably, of containing the metabolite or metabolites relevant for an intended use in a concentration essentially as it is found in a freshly drawn sample. More preferably, sufficient quality is the property of containing the metabolite or metabolites relevant for an intended use in a concentration essentially as it is found in a sample processed according to standard protocols. A sample being of sufficient quality allows for proper analysis, preferably because the composition is not altered with respect to the amounts of metabolites as well as the chemical nature of metabolites. Preferably, the standard protocol for blood product sample processing comprises withdrawal of a blood sample at room temperature; centrifugation of said blood sample within 60 min in a centrifuge at a controlled temperature of 18° C. to 22° C. to remove blood cells, preferably for 10 min to 15 min; transfer of the supernatant of the centrifugation (plasma) into a fresh container and storage of said plasma at a temperature of at most −80° C. for at most one year. Still more preferably, sufficient quality relates to the property of a sample of the present invention of not being affected by changes caused by any the confounding factors (i) prolonged time between phlebotomy (withdrawal of blood) and separation of plasma from blood cells, (ii) unsuited temperature between phlebotomy and separation of plasma from blood cells, (iii) prolonged time of storage of plasma, and (iv) increased temperature during storage of plasma. It is understood by the skilled person that different classes of metabolites, and also different members within one of these classes, differ in their tendency to change with time and temperature within a sample. E.g. proteins are generally more stable than RNAs; and a protein like an IgG will generally be more stable than a peptide hormone or a cytokine.

As will be understood by the skilled person, blood is sensitive to cooling since blood platelets become activated by chilling and this will change the metabolome and the proteome and other biomolecules of the corresponding plasma derived from these samples. Therefore, depending on the intended application, a cooling of blood may be disadvantageous and the definition of blood processing temperature is crucial in e.g. multi-center studies to avoid imbalances in study groups. As soon as the blood cells are removed from the plasma, cooling is advantageous in order to minimize enzymatic and/or chemical-oxidative pre-analytical effects on the samples. Preferred sample preparation protocols are known in the art and are described, e.g., in the references cited herein above, including, e.g. for proteomics the protocols reviewed in Rai & Vitzthum (loc. cit.). From the above, it will be understood that, preferably, the standard protocol may vary, depending on the intended use.

In a preferred embodiment, the intended use is use of a sample of the invention in metabolomics and/or in determining at least one biomarker, and sufficient quality relates to a composition of a sample which allows for a proper analysis of the metabolomic composition. In a more preferred embodiment, the intended use is use of a sample of the invention in metabolomics and/or in determining at least one biomarker, and sufficient quality relates to the property of a sample of the present invention of not being affected by changes caused by any one of the confounding factors (i) time between phlebotomy and initiation of removal of blood cells more than 60 min, (ii) unsuited temperature between phlebotomy and initiation of removal of blood cells or during removal of blood cells, (iii) storage of plasma at a temperature of more than 5° C. for more than 30 min, and (iv) storage of plasma for more than one year at a temperature of −80° C. or higher. For the storage of plasma, the approximation of the Arrhenius equation applies for other storage temperatures and storage times.

Conversely, the term “insufficient quality” relates to the property of a sample of the present invention of providing measurement values perturbed by sample processing, i.e., preferably, of not containing the metabolite or metabolites relevant for an intended use in a concentration essentially as it is found in a freshly drawn sample. More preferably, insufficient quality is the property of containing the metabolite or metabolites relevant for an intended use in a concentration deviating, preferably significantly deviating, from the concentration found in a sample processed according to standard protocols as described herein above. A sample being of insufficient quality may cause an improper analysis because the metabolic composition is altered with respect to the amounts of metabolites as well as the chemical nature of metabolites. Insufficient quality may be caused, preferably, by degradation of metabolites and/or chemical alterations of the said metabolites. Still more preferably, insufficient quality relates to the property of a sample of the present invention of being affected by changes caused by at least one of the confounding factors (i) prolonged time between phlebotomy and separation of plasma from blood cells, (ii) unsuited temperature between phlebotomy and separation of plasma from blood cells, (iii) prolonged time of storage of plasma, and (iv) increased temperature during storage of plasma. In a preferred embodiment, the intended use is use of a sample of the invention in metabolomics and/or in determining at least one biomarker, and insufficient quality relates to a composition of a sample which does not allow for a proper analysis of the metabolomic composition. In a more preferred embodiment, the intended use is use of a sample of the invention in metabolomics and/or in determining at least one biomarker, and insufficient quality relates to the property of a sample of the present invention of being affected by changes caused by at least one of the confounding factors (i) time between phlebotomy and initiation of removal of blood cells more than 60 min, (ii) unsuited temperature between phlebotomy and initiation of removal of blood cells or during removal of blood cells, (iii) storage of plasma at a temperature of more than 5° C. for more than 30 min, and (iv) storage of plasma for more than one year at a temperature of −80° or higher.

The term “assessing”, as used herein, relates to distinguishing between insufficient and sufficient quality of a blood product sample. Preferably, assessing relates to excluding that any of the confounding factors as described elsewhere herein has affected a sample, i.e., preferably, assessing relates to classifying a sample as acceptable, i.e. having sufficient quality, or not acceptable, i.e. having insufficient quality, for an intended use. In a further preferred embodiment, assessing further comprises distinguishing whether a sample was affected by a confounding factor related to blood processing, i.e., preferably, prolonged time between phlebotomy and separation of plasma from blood cells or increased temperature between phlebotomy and separation of plasma from blood cells, or whether said sample was affected by a confounding factor related to plasma processing and/or storage, i.e., preferably, prolonged time of storage of plasma or increased temperature during storage of plasma. As will be understood by those skilled in the art, such an assessment, although preferred to be, may usually not be correct for 100% of the samples investigated. The term, however, requires that a statistically significant portion of samples can be correctly assessed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, or 0.05.

Preferably, assessing includes classifying a sample according to more than two quality classes, e.g. the three quality classes high quality, medium quality, and low quality. More preferably, classification of the sample according to any of the methods described herein may even include (or result in) a more precise estimation on the processing conditions applied to respective sample; for example, preferably, with regard to blood processing related confounders, “high quality” may be described as centrifugation of the sample within a period of two hours after blood draw; “medium quality” may be described as centrifugation of the sample within a period of two to six hours after blood draw; and “low quality” may be described as centrifugation of the sample more than six hours after blood draw or an influence of platelet activation on the sample. With regard to plasma processing related confounders, “high quality” may be described as processing of the sample within a period of six or less hours at room temperature after centrifugation and laboratory bench processing time; “medium quality” may be described as processing of the sample within a period of six to 24 hours after centrifugation; and “low quality” may be described as processing of the sample within a period more than 24 hours after centrifugation. Preferably, classification of a sample comprises determining a quality score as specified herein below.

Preferably, in the case where a numerical quality assessment of a sample (a quality score) is desired, the values of the markers of a panel are categorized by comparison to pre-defined cut-offs and, preferably, are combined into a single value which is then scaled, for example, between 1 and 100.

More preferably, the metabolites are weighted due to their importance.

Most preferably, the individual numerical values of the markers of a panel are translated into a combined value by using multivariate models, for example a logistic regression model.

Moreover, as indicated above, the numerical quality assessment (quality score), preferably, is calculated separately (i) for blood processing related confounders (time and temperature between blood draw (phlebotomy) and separation of plasma from cells, e.g. by centrifugation of blood tubes) and (ii) for plasma processing related confounders (time and temperature of short- and long-term storage of blood plasma, either frozen or liquid). Marker assignment to confounders related to either blood-, or plasma processing is preferably done according to Table 3.

As used herein, the term “marker” relates to any chemical or mathematical entity serving as an indicator for quality as referred to in this specification. Preferably, the marker is a biomarker as specified herein below, i.e. preferably, the presence or absence of said biomarker; more preferably, the marker is an absolute or, preferably, relative concentration of a biomarker in a sample or a value derived therefrom in any standard mathematical calculation. Accordingly, the marker, preferably, is the intra-sample ratio of the concentrations of at least two biomarkers of the present invention.

The terms “marker panel” and “panel” relate to one of the specific marker combinations identified as panels in one of Tables 1 and 2 of the present invention. Preferably, a panel comprises at least three markers, of which at least one marker is suitable for indicating insufficient quality related to confounding factors related to blood processing, and of which at least a second marker is suitable for indicating insufficient quality related to confounding factors related to plasma processing.

The term “biomarker”, as used herein, refers to a chemical molecule serving as an indicator for quality as referred to in this specification. Preferably, said chemical molecule is a metabolite itself which is found in a sample of a subject. Moreover, the biomarker may also be a molecular species which is derived from said metabolite. In such a case, the actual metabolite will be chemically modified in the sample or during the determination process and, as a result of said modification, a chemically different molecular species, i.e. the analyte, will be the determined molecular species. It is to be understood that in such a case, the analyte represents the actual metabolite and has the same potential as an indicator for quality. Moreover, a biomarker according to the present invention is not necessarily corresponding to one molecular species. Rather, the biomarker may comprise stereoisomers or enantiomeres of a compound. Accordingly, e.g., the biomarker glycerol-3-phosphate preferably includes its stereoisomer glycerol-1-phosphate. Further, a biomarker can also represent the sum of isomers of a biological class of isomeric molecules. Said isomers preferably exhibit identical analytical characteristics in some cases and are, therefore, not distinguishable by various analytical methods including those applied in the accompanying Examples described below. However, the isomers will share at least identical sum formula parameters and, thus, in the case of, e.g., lipids an identical chain length and identical numbers of double bonds in the fatty acid and/or sphingo base moieties. Polar biomarkers can be, preferably, obtained by techniques referred to in this specification elsewhere and as described in Examples, below. Lipid biomarkers can be obtained in accordance with the present invention, preferably, as described in this specification elsewhere and, in particular, either as lipid fraction by separation of a sample after protein precipitation into an aqueous polar and an organic lipid phase by, e.g., a mixture of ethanol and dichloromethane as described in Examples, below. Alternatively or in addition, biomarkers may be enriched from the sample using solid phase extraction (SPE). Also included as a biomarker of the present invention is the presence or absence or, preferably, the concentration, of a chemical compound at least in part exogenously added to a sample, e.g. ethylenediaminetetraacetic acid (EDTA) or citrate, or a value mathematically derived thereof.

The term “matrix check”, as used herein, relates to ascertaining the type of anticoagulant or absence thereof. Preferably, matrix check is determining whether a sample comprises EDTA, citrate, or heparin as an anticoagulant or does not comprise an anticoagulant. More preferably, matrix check is ascertaining whether a sample type is an EDTA plasma sample, a citrate plasma sample, a heparin plasma sample or a serum sample. Accordingly, preferably, matrix check is verifying that the anticoagulant present in a sample is in accord with the anticoagulant intended to be present in said sample.

The term “metabolite”, as used herein, relates to at least one molecule of a specific metabolite up to a plurality of molecules of the said specific metabolite. It is to be understood further that a group of metabolites means a plurality of chemically different molecules wherein for each metabolite at least one molecule up to a plurality of molecules may be present. A metabolite in accordance with the present invention encompasses all classes of organic or inorganic chemical compounds including those being comprised by biological material such as organisms. Accordingly, preferably, the metabolite is a biological macromolecule, e.g. preferably, DNA, RNA, protein, or a fragment thereof. More preferably, the metabolite in accordance with the present invention is a small molecule compound. More preferably, in case a plurality of metabolites is envisaged, said plurality of metabolites representing a metabolome, i.e. the collection of metabolites being comprised by an organism, an organ, a tissue, a body fluid or a cell at a specific time and under specific conditions. In addition to the specific biomarkers recited in the specification, other biomarkers and/or indicators may be, preferably, determined as well in the methods of the present invention. Such biomarkers may include peptide or polypeptide biomarkers, e.g., those referred to in WO2012/170669, Liu et al. 2010, Anal Biochem 406: 105-115, or Fliniaux et al. 2011, Journal of Biomolecular NMR 51(4): 457-465).

In the method according to the present invention, values of at least the markers of at least one panel of Table 1 are determined. In a preferred embodiment, values of at least the markers of at least panel 3_a, 13_a, 15_a, 16_a, 1_a, 1_b, 10_a, 11_a, 12_a, 13_b, 14_a, 14_b, 17_b, 18_b, 19_a, 19_b, 2_b, 20_b, 3_b, 4_a, 5_a, 5_b, 6_a, 7_a, 7_b, 8_b, 1, 2, 3, 4, 5, 6, 7, 8, 10, 11, 12, 13, 14, 15, 16, 17, 18, 19, 20, 3_m, 6_m, 10_m, 15_m, 16_m, 17_m, 18_m, 19_m, or 20_m are determined.

Preferably, at least the markers of panel 2_b of Table 1 are determined, which can preferably be determined by enzymatic methods; or at least the markers of panel 4_a of Table 1 are determined, which can preferably be analyzed by a combination of SPE with LC and mass spectrometry, in particular with SPE-LC-MS/MS method focusing on eicosanoids; or at least the markers of panel 5_a or 5_b of table 1 are determined, which can preferably be analyzed from the lipid fraction by a combination of LC with mass spectrometry, in particular by LC-MS/MS; or at least the markers of panel 6_a of Table 1 are determined, which can preferably be analyzed from the polar fraction by a combination of LC with mass spectrometry, in particular by LC-MS/MS; or at least the markers of panel 7a or 7b—in particular of panel 7_a—of Table 1 are determined, which can preferably be analyzed by a combination of SPE with LC and mass spectrometry, in particular with SPE-UPLC-MS/MS method focusing on sphingoids; or at least the markers of panel 12_a of Table 1 are determined, which can preferably be analyzed from the lipid fraction; or at least the markers of panel 14_a or 14_b of Table 1 are determined, focusing on confounders related to time and temperature of short- and long-term storage of blood plasma (either frozen, or liquid); or, preferably, at least the markers of panel 1_a, 10_a, 11_a, 13_a, 13_b, 15_a, 16_a, 18_b, 19_a, 19_b, 20_b, 3_a, or 3_b of Table 1 are determined, which can preferably be analyzed from the polar fraction by a combination of GC and mass spectrometry, in particular with GC-MS; or, preferably, at least the markers of panel 1_a, 10_a, 11_a, 13_a, 13_b, 15_a, 16_a, 18_b, 19_a, 19_b, 20_b, 3_a, or 3_b of Table 1 are determined, which can preferably be analyzed from the polar fraction by a combination of LC and mass spectrometry, in particular with LC-MS/MS. Most preferably, the values of at least the markers of at least one of panels 3_a, 3_b, 10_a, 11_a, 13_a, 13_b, 15_a, 16_a, 17_b, 18_b, 19_a, 19_b, or 20_b of Table 1 are determined in the method of the present invention.

TABLE 1
Marker panels of the present invention; Panel numbers, markers comprised therein,
and direction of change indicative of insufficient quality are indicated.

| Panel Number | Marker | Direction |
|---|---|---|
| 3_a | Ornithine | up |
| | Glycerol-3-phosphate | up |
| | Glycerate | up |
| 13_a | Glycerol-3-phosphate | up |
| | Glycerate | up |
| | Hypoxanthine | up |
| 15_a | Ornithine | up |
| | Glycerol-3-phosphate | up |
| | Hypoxanthine | up |
| 16_a | Ornithine | up |
| | Glycerate | up |
| | Hypoxanthine | up |
| 1_a | Ornithine | up |
| | Arginine | down |
| | Hypoxanthine | up |
| 1_b | Ornithine | up |
| | Arginine | down |
| | Hypoxanthine | up |
| | Sphingadienine-1-phosphate (d18:2) | up |
| 10_a | Ornithine | up |
| | Glycerol-3-phosphate | up |
| | Glycerate | up |
| | Hypoxanthine | up |
| 11_a | Glycerate | up |
| | Hypoxanthine | up |
| | Glutamate/Glutamine intra-sample ratio | up |
| 12_a | Cholesterylester hydroperoxide (C18:2-9-OOH) | up |
| | Sphingosine (d18:1) | down |
| | Sphingosine-1-phosphate (d18:1) | up |
| 13_b | Glutamine | down |
| | Glutamate | up |
| | Hypoxanthine | up |
| 14_a | Glycerol-3-phosphate | up |
| | Glycerate | up |
| | Glutamate/Glutamine intra-sample ratio | up |
| 14_b | 3-Phosphoglycerate (3-PGA) | up |
| | 3,4-Dihydroxyphenylacetic acid (DOPAC) | down |
| | 3,4-Dihydroxyphenylglycol (DOPEG) | down |
| 17_b | Ornithine | up |
| | Hypoxanthine | up |
| | 3,4-Dihydroxyphenylacetic acid (DOPAC) | down |
| | Glutamate/Glutamine intra-sample ratio | up |
| 18_b | Arginine | down |
| | Glycerol-3-phosphate | up |
| | Threonic acid | up |
| | Hypoxanthine | up |
| 19_a | Glycerol-3-phosphate | up |
| | Glycerate | up |
| | Hypoxanthine | up |
| | Ornithine/Arginine intra-sample ratio | up |
| 19_b | Glycerate | up |
| | Glutamate/Glutamine intra-sample ratio | up |
| | Ornithine/Arginine intra-sample ratio | up |
| 2_b | Glutamine | down |
| | Glycerol-3-phosphate | up |
| | Glutamate | up |
| | Hypoxanthine | up |
| 20_b | Glycerol-3-phosphate | up |
| | Threonic acid | up |
| | Glutamate/Glutamine intra-sample ratio | up |
| 3_b | Ornithine | up |
| | Hypoxanthine | up |
| | Glutamate/Glutamine intra-sample ratio | up |
| 4_a | 15-Hydroxyeicosatetraenoic acid (C20:cis[5,8,11,13]4) | up |
| | 5-Hydroxyeicosatetraenoic acid | up |
| | (C20:trans[6]cis[8,11,14]4) (5-HETE) | |
| | 12-Hydroxyeicosatetraenoic acid (C20:cis[5,8,10,14]4) | down |
| 5_a | Phosphatidylcholine hydroperoxide (C18:0,C1 8:2-OOH) | up |
| | Triacylgyceride hydroperoxide (C16:0,C18:1,C18:3-OOH) | up |
| | Cholesterylester hydroperoxide (C18:2-9-OOH) | up |
| 5_b | Lysophosphatidylcholine (C18:1) | up |
| | Phosphatidylcholine hydroperoxide (C18:0,C18:2-OOH) | up |
| | Cholesterylester hydroperoxide (C18:2-9-OOH) | up |
| 6_a | Arginine | down |
| | Glycerol-3-phosphate | up |
| | Glutamate/Glutamine intra-sample ratio | up |
| 7_a | Sphingosine (d16:1) | down |
| | Sphingadienine (d18:2) | down |
| | Sphingosine-1-phosphate (d16:1) | up |
| | Sphingadienine-1-phosphate (d18:2) | up |
| 7_b | Sphingadienine (d18:2) | down |
| | Sphingosine (d18:1) | down |
| | Sphingadienine-1-phosphate (d18:2) | up |
| | Sphingosine-1-phosphate (d18:1) | up |
| 8_b | Glycerate | up |
| | Phosphatidylcholine hydroperoxide (C16:0,C18:1-OOH) | up |
| | Cholesterylester hydroperoxide (C18:2-13-OOH) | up |

More preferably, at least the amounts of the biomarkers (i) glycerol-3-phosphate, glycerate, and ornithine; (ii) glycerol-3-phosphate, glycerate, and hypoxanthine; (iii) glycerol-3-phosphate, ornithine, and hypoxanthine; or (iv) glycerate, ornithine, and hypoxanthine are determined in the method of the present invention. It is understood by the skilled person that determining the amount of a biomarker may be followed by calculating the value of a further marker therefrom; e.g., preferably, the amount of ornithine determined may be used to calculate an ornithine/arginine intra-sample ratio as a marker.

Still more preferably, at least the values of at least the markers of at least one panel of Table 2 are determined. The panels of Table 1 are sub-panels of the panels indicated in Table 2; the numbering of a sub-panel indicates the panel of Table 2 it is derived from. Accordingly, sub-panels X_a and/or X_b, with X=1, 2, 3, 4, 5, 6, 7, 8, 10, 11, 12, 13, 14, 15, 16, 17, 18, 19, or 20, are derived from panel X in Table 2. It is, however, understood that sub-panels may comprise markers also comprised in further panels of Table 2.

Preferably, at least the markers of panel 1 of table 2 are determined, focusing on confounders related to time and temperature between phlebotomy and separation of plasma from cells; or at least the markers of panel 2 of table 2 are determined, which preferably can be determined by enzymatic methods; or at least the markers of panel 4 of Table 2 are determined, which can preferably be analyzed by a combination of SPE with LC and mass spectrometry, in particular by a SPE-LC-MS/MS method focusing on eicosanoids; or at least the markers of panel 5 of table 2 are determined, which can preferably be analyzed from the lipid fraction by a combination of LC with mass spectrometry, in particular with LC-MS/MS; or at least the markers of panel 6 of table 2 are determined, which can preferably be analyzed from the polar fraction by a combination of LC with mass spectrometry, in particular with LC-MS/MS; or at least the markers of panel 7 of Table 2 are determined, which can preferably be analyzed by a combination of SPE with LC and mass spectrometry, in particular with a SPE-UPLC-MS/MS method focusing on sphingoids; or at least the markers of panel 8 of Table 2 are determined, focusing on confounders related to time and temperature of long-term storage of frozen blood plasma; or at least the markers of panel 12 of Table 2 are determined, which can preferably be analyzed from the lipid fraction; or at least the markers of panel 14 of Table 2 are determined, focusing on confounders related to time and temperature of short- and long-term storage of blood plasma (either frozen, or liquid), or, preferably, at least the markers of panel 3, 15, 16, 18, 19, or 20 of Table 2 are determined, which can preferably be analyzed from the polar fraction by a combination of GC and mass spectrometry, in particular with GC-MS; or, preferably, at least the markers of panel 3, 15, 16, 18, 19, or 20 of Table 2 are determined, which can preferably be analyzed from the polar fraction by a combination of LC and mass spectrometry, in particular with LC-MS/MS. Most preferably, the values of at least the markers of at least one of panels 3, 10, 11, 13, 15, 16, 17, 18, 19, or 20 of Table 2 are determined in the method of the present invention.

TABLE 2
Further improved marker panels of the present invention; Panel numbers, markers
comprised therein, and direction of change indicative
of insufficient quality are indicated.

| Panel Number | Marker | Direction |
|---|---|---|
| 1 | Hypoxanthine | up |
| | Ornithine | up |
| | Arginine | down |
| | Ornithine/Arginine intra-sample ratio | up |
| | Ribose | up |
| | Sphingosine (d18:1) | down |
| | Sphingosine-1-phosphate (d18:1) | up |
| | Glucose | down |
| | Lactate | up |
| | Sphingosine (d16:1) | down |
| | Sphingosine-1-phosphate (d16:1) | up |
| | Taurine | down |
| | 12-Hydroxyeicosatetraenoic acid (C20:cis[5,8,10,14]4) | down |
| | Lactate/Glucose intra-sample ratio | up |
| | Citrulline | up |
| | 12-Hydroxyheptadecatrienoic acid (C17:[5,8,10]3) | down |
| | 13-Hydroxyactadecadienoic acid (13-HODE) | up |
| | (C18:cis[9]trans[11]2) | |
| | 5-Hydroxyeicosatetraenoic acid (C20:trans[6]cis[8,11,14]4) | up |
| | (5-HETE) | |
| | 9-Hydroxyoctadecadienoic acid (9-HODE) | up |
| | (C18:trans[10]cis[12]2) | |
| | Glucose-6-phosphate | up |
| | Pentoses | up |
| | Serotonin (5-HT) | down |
| | Sphingadienine (d18:2) | down |
| | Sphingadienine-1-phosphate (d18:2) | up |
| | Sphingosine-1-phosphate (d17:1) | up |
| | Thromboxane B2 | down |
| 2 | Glycerate | up |
| | Glycerol-3-phosphate | up |
| | Hypoxanthine | up |
| | 3-Phosphoglycerate (3-PGA) | up |
| | Glutamate | up |
| | Glutamine | down |
| | Ornithine | up |
| | Cysteine | down |
| | 3,4-Dihydroxyphenylacetic acid (DOPAC) | down |
| | 3,4-Dihydroxyphenylglycol (DOPEG) | down |
| | Sphingosine (d18:1) | down |
| | Sphingosine-1-phosphate (d18:1) | up |
| | Cholesterylester hydroperoxide (C18:2-9-OOH) | up |
| | Sphingosine (d16:1) | down |
| | Sphingosine-1-phosphate (d16:1) | up |
| | Phosphatidylcholine hydroperoxide (C18:0,C18:2-OOH) | up |
| | Triacylgyceride hydroperoxide (C16:0,C18:1,C18:3-OOH) | up |
| 3 | Glycerate | up |
| | Glycerol-3-phosphate | up |
| | Glutamate/Glutamine intra-sample ratio | up |
| | Hypoxanthine | up |
| | 3-Phosphoglycerate (3-PGA) | up |
| | Glutamate | up |
| | Glutamine | down |
| | Ornithine | up |
| | Cysteine | down |
| | Threonic acid | up |
| | Arginine | down |
| | Asparagine | down |
| | Aspartate/Asparagine intra-sample ratio | up |
| | Ornithine/Arginine intra-sample ratio | up |
| | Ribose | up |
| | Cystine | down |
| | Glucose | down |
| | Lactate | up |
| | Taurine | down |
| | Lactate/Glucose intra-sample ratio | up |
| | Citrulline | up |
| | Maltose | up |
| | Alanine | up |
| | Maltotriose | up |
| | Uric acid | up |
| 4 | 12-Hydroxyeicosatetraenoic acid (C20:cis[5,8,10,14]4) | down |
| | 15-Hydroxyeicosatetraenoic acid (C20:cis[5,8,11,13]4) | up |
| | 12-Hydroxyheptadecatrienoic acid (C17:[5,8,10]3) | up |
| | 13-Hydroxyoctadecadienoic acid (13-HODE) | up |
| | (C18:cis[9]trans[11]2) | |
| | 5-Hydroxyeicosatetraenoic acid (C20:trans[6]cis[8,11,14]4) | up |
| | (5-HETE) | |
| | 9-Hydroxyoctadecadienoic acid (9-HODE) | up |
| | (C18:trans[10]cis[12]2) | |
| | Thromboxane B2 | up |
| | 11-Hydroxyeicosatetraenoic acid (C20:cis[5,8,12,14]4) | up |
| | 8,9-Dihydroxyeicosatrienoic acid (C20:cis[5,11,14]3) | up |
| | 8-Hydroxyeicosatetraenoic acid (C20:trans[5]cis[9,11,14]4) | up |
| | (8-HETE) | |
| | Prostaglandin D2 | up |
| | Prostaglandin E2 | up |
| 5 | Cholesterylester hydroperoxide (C18:2-9-OOH) | up |
| | Phosphatidylcholine hydroperoxide (C18:0,C18:2-OOH) | up |
| | Triacylgyceride hydroperoxide (C16:0,C18:1,C18:3-OOH) | up |
| | Cholesterylester hydroperoxide (C20:4-OOH) | up |
| | Lysophosphatidylcholine (C18:0) | up |
| | Phosphatidylcholine hydroperoxide (C16:0,C18:1-OOH) | up |
| | Ceramide (d18:1,C24:0) | up |
| | Cholesterylester hydroperoxide (C18:2-13-OOH) | up |
| | Lysophosphatidylcholine (C17:0) | up |
| | Lysophosphatidylcholine (C18:1) | up |
| | Lysophosphatidylcholine (C20:4) | up |
| | Phosphatidylcholine hydroperoxide (C16:0,C18:2-OOH) | up |
| | Triacylgyceride hydroperoxide (C16:0,C18:1,C18:2-OOH) | up |
| | Triacylgyceride hydroperoxide (C16:0,C18:1,C20:4-OOH) | up |
| | Triacylgyceride hydroperoxide (C16:0,C18:2,C18:2-OOH) | up |
| | Triacylgyceride hydroperoxide (C18:1,18:2,C18:2-OOH) | up |
| | Triacylgyceride hydroperoxide (C18:1,C18:1,C18:3-OOH) | up |
| 6 | Glycerol-3-phosphate | up |
| | Glutamate/Glutamine intra-sample ratio | up |
| | Glutamate | up |
| | Glutamine | down |
| | Arginine | down |
| | Asparagine | down |
| | Aspartate/Asparagine intra-sample ratio | up |
| | Glucose-6-phosphate | up |
| | Pentoses | up |
| | Creatinine | up |
| 7 | Sphingosine (d18:1) | down |
| | Sphingosine-1-phosphate (d18:1) | up |
| | Sphingosine (d16:1) | down |
| | Sphingosine-1-phosphate (d16:1) | up |
| | Sphingadienine (d18:2) | down |
| | Sphingadienine-1-phosphate (d18:2) | up |
| | Sphingosine-1-phosphate (d17:1) | up |
| 8 | Glycerate | up |
| | Glycerol-3-phosphate | up |
| | Glutamate/Glutamine intra-sample ratio | up |
| | 3-Phosphoglycerate (3-PGA) | up |
| | Cysteine | down |
| | Threonic acid | up |
| | 3,4-Dihydroxyphenylacetic acid (DOPAC) | down |
| | 3,4-Dihydroxyphenylglycol (DOPEG) | down |
| | Aspartate/Asparagine intra-sample ratio | up |
| | Cholesterylester hydroperoxide (C18:2-9-OOH) | up |
| | Cystine | down |
| | Phosphatidylcholine hydroperoxide (C18:0,C18:2-OOH) | up |
| | Triacylgyceride hydroperoxide (C16:0,C18:1,C18:3-OOH) | up |
| | Cholesterylester hydroperoxide (C20:4-OOH) | up |
| | Lysophosphatidylcholine (C18:0) | up |
| | Phosphatidylcholine hydroperoxide (C16:0,C18:1-OOH) | up |
| | Cholesterylester hydroperoxide (C18:2-13-OOH) | up |
| | Phosphatidylcholine hydroperoxide (C16:0,C18:2-OOH) | up |
| | Triacylgyceride hydroperoxide (C16:0,C18:1,C18:2-OOH) | up |
| | Triacylgyceride hydroperoxide (C16:0,C18:1,C20:4-OOH) | up |
| | Triacylgyceride hydroperoxide (C16:0,C18:2,C18:2-OOH) | up |
| | Triacylgyceride hydroperoxide (C18:1,18:2,C18:2-OOH) | up |
| | Triacylgyceride hydroperoxide (C18:1,C18:1,C18:3-OOH) | up |
| 10 | Glycerate | up |
| | Glycerol-3-phosphate | up |
| | Hypoxanthine | up |
| | 3-Phosphoglycerate (3-PGA) | up |
| | Glutamate | up |
| | Glutamine | down |
| | Ornithine | up |
| | Cysteine | down |
| | Threonic acid | up |
| | 3,4-Dihydroxyphenylacetic acid (DOPAC) | down |
| | Arginine | down |
| | 3,4-Dihydroxyphenylglycol (DOPEG) | down |
| | Asparagine | down |
| | Ribose | up |
| | Cystine | down |
| | Glucose | down |
| | Lactate | up |
| | Taurine | down |
| | Citrulline | up |
| | Maltose | up |
| 11 | Glycerate | up |
| | Glutamate/Glutamine intra-sample ratio | up |
| | Hypoxanthine | up |
| | 3-Phosphoglycerate (3-PGA) | up |
| | Glutamate | up |
| | Glutamine | down |
| | Cysteine | down |
| | 3,4-Dihydroxyphenylacetic acid (DOPAC) | down |
| | Sphingosine (d18:1) | down |
| | Sphingosine-1-phosphate (d18:1) | up |
| | Cholesterylester hydroperoxide (C18:2-9-OOH) | up |
| 12 | Sphingosine (d18:1) | down |
| | Sphingosine-1-phosphate (d18:1) | up |
| | Cholesterylester hydroperoxide (C18:2-9-OOH) | up |
| | Sphingosine (d16:1) | down |
| | Sphingosine-1-phosphate (d16:1) | up |
| | 12-Hydroxyeicosatetraenoic acid (C20:cis[5,8,10,14]4) | down |
| | Phosphatidylcholine hydroperoxide (C18:0,C18:2-OOH) | up |
| | Triacylgyceride hydroperoxide (C16:0,C18:1,C18:3-OOH) | up |
| | 15-Hydroxyeicosatetraenoic acid (C20:cis[5,8,11,13]4) | up |
| 13 | Glycerate | up |
| | Glycerol-3-phosphate | up |
| | Hypoxanthine | up |
| | 3-Phosphoglycerate (3-PGA) | up |
| | Glutamate | up |
| | Glutamine | down |
| | Ornithine | up |
| | Cysteine | down |
| | 3,4-Dihydroxyphenylacetic acid (DOPAC) | down |
| | 3,4-Dihydroxyphenylglycol (DOPEG) | down |
| 14 | Glycerate | up |
| | Glycerol-3-phosphate | up |
| | Glutamate/Glutamine intra-sample ratio | up |
| | 3-Phosphoglycerate (3-PGA) | up |
| | Glutamate | up |
| | Glutamine | down |
| | Cysteine | down |
| | Threonic acid | up |
| | 3,4-Dihydroxyphenylacetic acid (DOPAC) | down |
| | 3,4-Dihydroxyphenylglycol (DOPEG) | down |
| | Aspartate/Asparagine intra-sample ratio | up |
| | Cystine | down |
| | Lysophosphatidylcholine (C18:0) | up |
| | 3,4-Dihydroxyphenylalanine (DOPA) | down |
| | Alanine | up |
| | Ceramide (d18:1,C24:0) | up |
| | Lysophosphatidylcholine (C17:0) | up |
| | Lysophosphatidylcholine (C18:1) | up |
| | Lysophosphatidylcholine (C20:4) | up |
| | Adrenaline (Epinephrine) | down |
| | Noradrenaline (Norepinephrine) | down |
| 15 | Glycerate | up |
| | Glycerol-3-phosphate | up |
| | Glutamate/Glutamine intra-sample ratio | up |
| | Hypoxanthine | up |
| | 3-Phosphoglycerate (3-PGA) | up |
| | Glutamate | up |
| | Glutamine | down |
| | Ornithine | up |
| | Arginine | down |
| | Asparagine | down |
| | Ornithine/Arginine intra-sample ratio | up |
| | Ribose | up |
| | Glucose | down |
| | Lactate | up |
| | Taurine | down |
| 16 | Glycerate | up |
| | Glycerol-3-phosphate | up |
| | Glutamate/Glutamine intra-sample ratio | up |
| | Hypoxanthine | up |
| | 3-Phosphoglycerate (3-PGA) | up |
| | Glutamate | up |
| | Glutamine | down |
| | Ornithine | up |
| | Cysteine | down |
| | Threonic acid | up |
| | Arginine | down |
| | Asparagine | down |
| | Aspartate/Asparagine intra-sample ratio | up |
| | Ornithine/Arginine intra-sample ratio | up |
| | Ribose | up |
| | Cystine | down |
| | Glucose | down |
| | Lactate | up |
| | Taurine | down |
| | Lactate/Glucose intra-sample ratio | up |
| | Maltose | up |
| 17 | Glycerate | up |
| | Glycerol-3-phosphate | up |
| | Glutamate/Glutamine intra-sample ratio | up |
| | Hypoxanthine | up |
| | 3-Phosphoglycerate (3-PGA) | up |
| | Glutamate | up |
| | Glutamine | down |
| | Ornithine | up |
| | 3,4-Dihydroxyphenylacetic acid (DOPAC) | down |
| | 3,4-Dihydroxyphenylglycol (DOPEG) | down |
| | Asparagine | down |
| | Aspartate/Asparagine intra-sample ratio | up |
| | Ornithine/Arginine intra-sample ratio | up |
| | Ribose | up |
| | Sphingosine (d18:1) | down |
| | Sphingosine-1-phosphate (d18:1) | up |
| | Sphingosine (d16:1) | down |
| | Sphingosine-1-phosphate (d16:1) | up |
| | 12-Hydroxyeicosatetraenoic acid (C20:cis[5,8,10,14]4) | down |
| | Lactate/Glucose intra-sample ratio | up |
| | 15-Hydroxyeicosatetraenoic acid (C20:cis[5,8,11,13]4) | up |
| | Cholesterylester hydroperoxide (C20:4-OOH) | up |
| | Phosphatidylcholine hydroperoxide (C16:0,C18:1-OOH) | up |
| | 3,4-Dihydroxyphenylalanine (DOPA) | down |
| | Serotonin (5-HT) | down |
| 18 | Glycerate | up |
| | Glycerol-3-phosphate | up |
| | Glutamate/Glutamine intra-sample ratio | up |
| | Hypoxanthine | up |
| | Ornithine | up |
| | Threonic acid | up |
| | Arginine | down |
| 19 | Glycerate | up |
| | Glycerol-3-phosphate | up |
| | Glutamate/Glutamine intra-sample ratio | up |
| | Hypoxanthine | up |
| | Threonic acid | up |
| | Ornithine/Arginine intra-sample ratio | up |
| 20 | Glycerate | up |
| | Glycerol-3-phosphate | up |
| | Glutamate/Glutamine intra-sample ratio | up |
| | Hypoxanthine | up |
| | Ornithine | up |
| | Threonic acid | up |

In a preferred embodiment, in addition to the markers of at least one panel of table 1 or 2, at least one, more preferably at least two, most preferably all three of the biomarkers aspartate, citrate, and ethylenediaminetetraacetic acid (EDTA) (a combination of all three is also referred to as “panel number 9”) are determined, preferably as a matrix check as specified herein above. Preferably, presence of EDTA in a sample indicates that EDTA was used as an anticoagulant, whereas increased amounts of citrate indicate that citrate was used as an anticoagulant. Moreover, an increase in aspartate indicates that no inhibitor of coagulation was used. Accordingly, the biomarkers aspartate, citrate, and EDTA allow differentiating whether a sample was compromised by factors related to collection tube selection. Thus, preferably, the respective directions of change if an EDTA plasma is used as a reference are: EDTA down and/or citrate up and/or aspartate up, and, wherein, preferably, changes in said directions indicate that said sample is not an EDTA sample.

Accordingly, in a preferred embodiment, at least the markers of panel 3_a, 13_a, 15_a, 16_a, 1_a, 1_b, 10_a, 11_a, 12_a, 13_b, 14_a, 14_b, 17_b, 18_b, 19_a, 19_b, 2_b, 20_b, 3_b, 4_a, 5_a, 5_b, 6_a, 7_a, 7_b, or 8_b of Table 1 are determined in combination with the markers of panel 9. In a further preferred embodiment, at least the markers of panel 1, 2, 3, 4, 5, 6, 7, 8, 10, 11, 12, 13, 14, 15, 16, 17, 18, 19, or 20 of Table 2 are determined in combination with the markers of panel 9. In a more preferred embodiment, at least the markers of panel 6_m or 17_m of Table 2a are determined in combination with the markers of panel 9. In a still more preferred embodiment, the reference is a sample comprising EDTA as anticoagulant, more preferably, the reference is EDTA plasma, and, preferably, at least the markers of at least one of the panels 3_m, 10_m, 16_m, 18_m, 19_m, or 20_m of Table 2a are determined in the method of the present invention. In another preferred embodiment, at least the markers of panel 6_m of Table 2a, which can preferably be analyzed from the polar fraction by LC-MS/MS, are determined; or at least the markers of panel 15_m of Table 2a are determined; or at least the markers of panel 17_m of Table 2a, which has the highest performance (AUC-values), are determined in the method of the present invention. In another preferred embodiment, at least the markers of panel 3_m, 15_m, 16_m, 18_m, 19_m, or 20_m of Table 2a are determined, which can preferably be analyzed from the polar fraction by a combination of GC and mass spectrometry, in particular with GC-MS; or, preferably, at least the markers of panel 3_m, 15_m, 16_m, 18_m, 19_m, or 20_m of Table 2a are determined, which can preferably be analyzed from the polar fraction by a combination of LC and mass spectrometry, in particular with LC-MS/MS.

TABLE 2a
Further improved marker panels of the present invention with additional inclusion of
“matrix check” markers (matrix check markers according to Panel 9);
Panel numbers, markers comprised therein, and direction of change indicative of
insufficient quality are indicated.

| Panel Number | Marker | Direction |
|---|---|---|
| 3_m | Glycerate | up |
| | Glycerol-3-phosphate | up |
| | Glutamate/Glutamine intra-sample ratio | up |
| | Hypoxanthine | up |
| | 3-Phosphoglycerate (3-PGA) | up |
| | Aspartate | up |
| | Glutamate | up |
| | Glutamine | down |
| | Ornithine | up |
| | Citrate | up |
| | Cysteine | down |
| | Ethylenediaminetetraacetic acid (EDTA) | down |
| | Threonic acid | up |
| | Arginine | down |
| | Asparagine | down |
| | Aspartate/Asparagine intra-sample ratio | up |
| | Ornithine/Arginine intra-sample ratio | up |
| | Ribose | up |
| | Cystine | down |
| | Glucose | down |
| | Lactate | up |
| | Taurine | down |
| | Lactate/Glucose intra-sample ratio | up |
| | Citrulline | up |
| | Maltose | up |
| | Alanine | up |
| | Maltotriose | up |
| | Uric acid | up |
| 6_m | Glycerol-3-phosphate | up |
| | Glutamate/Glutamine intra-sample ratio | up |
| | Aspartate | up |
| | Glutamate | up |
| | Glutamine | down |
| | Citrate | up |
| | Arginine | down |
| | Asparagine | down |
| | Aspartate/Asparagine intra-sample ratio | up |
| | Glucose-6-phosphate | up |
| | Pentoses | up |
| | Creatinine | up |
| 10_m | Glycerate | up |
| | Glycerol-3-phosphate | up |
| | Hypoxanthine | up |
| | 3-Phosphoglycerate (3-PGA) | up |
| | Aspartate | up |
| | Glutamate | up |
| | Glutamine | down |
| | Ornithine | up |
| | Citrate | up |
| | Cysteine | down |
| | Ethylenediaminetetraacetic acid (EDTA) | down |
| | Threonic acid | up |
| | 3,4-Dihydroxyphenylacetic acid (DOPAC) | down |
| | Arginine | down |
| | 3,4-Dihydroxyphenylglycol (DOPEG) | down |
| | Asparagine | down |
| | Ribose | up |
| | Cystine | down |
| | Glucose | down |
| | Lactate | up |
| | Taurine | down |
| | Citrulline | up |
| | Maltose | up |
| 15_m | Glycerate | up |
| | Glycerol-3-phosphate | up |
| | Glutamate/Glutamine intra-sample ratio | up |
| | Hypoxanthine | up |
| | 3-Phosphoglycerate (3-PGA) | up |
| | Aspartate | up |
| | Glutamate | up |
| | Glutamine | down |
| | Ornithine | up |
| | Citrate | up |
| | Ethylenediaminetetraacetic acid (EDTA) | down |
| | Arginine | down |
| | Asparagine | down |
| | Ornithine/Arginine intra-sample ratio | up |
| | Ribose | up |
| | Glucose | down |
| | Lactate | up |
| | Taurine | down |
| 16_m | Glycerate | up |
| | Glycerol-3-phosphate | up |
| | Glutamate/Glutamine intra-sample ratio | up |
| | Hypoxanthine | up |
| | 3-Phosphoglycerate (3-PGA) | up |
| | Aspartate | up |
| | Glutamate | up |
| | Glutamine | down |
| | Ornithine | up |
| | Citrate | up |
| | Cysteine | down |
| | Ethylenediaminetetraacetic acid (EDTA) | down |
| | Threonic acid | up |
| | Arginine | down |
| | Asparagine | down |
| | Aspartate/Asparagine intra-sample ratio | up |
| | Ornithine/Arginine intra-sample ratio | up |
| | Ribose | up |
| | Cystine | down |
| | Glucose | down |
| | Lactate | up |
| | Taurine | down |
| | Lactate/Glucose intra-sample ratio | up |
| | Maltose | up |
| 17_m | Glycerate | up |
| | Glycerol-3-phosphate | up |
| | Glutamate/Glutamine intra-sample ratio | up |
| | Hypoxanthine | up |
| | 3-Phosphoglycerate (3-PGA) | up |
| | Aspartate | up |
| | Glutamate | up |
| | Glutamine | down |
| | Ornithine | up |
| | 3,4-Dihydroxyphenylacetic acid (DOPAC) | down |
| | 3,4-Dihydroxyphenylglycol (DOPEG) | down |
| | Asparagine | down |
| | Aspartate/Asparagine intra-sample ratio | up |
| | Ornithine/Arginine intra-sample ratio | up |
| | Ribose | up |
| | Sphingosine (d18:1) | down |
| | Sphingosine-1-phosphate (d18:1) | up |
| | Sphingosine (d16:1) | down |
| | Sphingosine-1-phosphate (d16:1) | up |
| | 12-Hydroxyeicosatetraenoic acid (C20:cis[5,8,10,14]4) | down |
| | Lactate/Glucose intra-sample ratio | up |
| | 15-Hydroxyeicosatetraenoic acid (C20:cis[5,8,11,13]4) | up |
| | Cholesterylester hydroperoxide (C20:4-OOH) | up |
| | Phosphatidylcholine hydroperoxide (C16:0,C18:1-OOH) | up |
| | 3,4-Dihydroxyphenylalanine (DOPA) | down |
| | Serotonin (5-HT) | down |
| 18_m | Glycerate | up |
| | Glycerol-3-phosphate | up |
| | Glutamate/Glutamine intra-sample ratio | up |
| | Hypoxanthine | up |
| | Aspartate | up |
| | Ornithine | up |
| | Citrate | up |
| | Ethylenediaminetetraacetic acid (EDTA) | down |
| | Threonic acid | up |
| | Arginine | down |
| 19_m | Glycerate | up |
| | Glycerol-3-phosphate | up |
| | Glutamate/Glutamine intra-sample ratio | up |
| | Hypoxanthine | up |
| | Aspartate | up |
| | Citrate | up |
| | Ethylenediaminetetraacetic acid (EDTA) | down |
| | Threonic acid | up |
| | Ornithine/Arginine intra-sampie ratio | up |
| 20_m | Glycerate | up |
| | Glycerol-3-phosphate | up |
| | Glutamate/Glutamine intra-sample ratio | up |
| | Hypoxanthine | up |
| | Aspartate | up |
| | Ornithine | up |
| | Citrate | up |
| | Ethylenediaminetetraacetic acid (EDTA) | down |
| | Threonic acid | up |

The term “sample”, as used herein, refers to samples comprising biological material and, in particular, the biomarkers as indicated herein above. Preferably, a sample in accordance with the present invention is a sample from a body fluid, preferably, lacrimal fluid, milk, saliva or urine, or a sample derived, e.g., by biopsy, from cells, tissues or organs. More preferably, the sample is blood or a blood product, most preferably, a plasma sample. The aforementioned samples can be derived from a subject as specified elsewhere herein. Techniques for obtaining the aforementioned different types of biological samples are well known in the art. For example, blood samples may be obtained by blood taking while tissue or organ samples are to be obtained, e.g., by biopsy. Preferably, the sample is a whole blood, serum, or plasma sample. In a preferred embodiment, the sample is a blood plasma sample, preferably a citrate plasma sample or an EDTA plasma sample. In another preferred embodiment, the sample is a blood serum sample.

The aforementioned samples are, preferably, pre-treated before they are used for the method of the present invention. As described in more detail below, said pre-treatment may include treatments required to release or separate the compounds or to remove excessive material or waste. Furthermore, pre-treatments may aim at sterilizing samples and/or removing contaminants such as undesired cells, bacteria or viruses. Suitable techniques comprise centrifugation, extraction, fractioning, ultrafiltration, and protein precipitation followed by filtration and purification and/or enrichment of compounds. Moreover, other pre-treatments are, preferably, carried out in order to provide the compounds in a form or concentration suitable for compound analysis. For example, if gas-chromatography coupled mass spectrometry is used in the method of the present invention, it may be required to derivatize the compounds prior to the said gas chromatography. Suitable and necessary pre-treatments also depend on the means used for carrying out the method of the invention and are well known to the person skilled in the art. In a preferred embodiment, a buffer compound, preferably in aqueous solution, is added to the sample. Buffer compounds, in particular neutral buffer compounds are, in principle, known to the skilled person. In a further preferred embodiment, protein comprised in the sample is precipitated, preferably by the addition of an appropriate organic solvent. Appropriate organic solvents for precipitating proteins are known in the art. In a further preferred embodiment, at least one phase-separating agent is added to the sample to enable separation of a polar phase and a lipophilic phase, preferably by sample centrifugation Pre-treated samples as described before are also comprised by the term “sample” as used in accordance with the present invention.

In a preferred embodiment, a polar and a lipophilic phase are obtained from the sample according to the aforementioned steps. In a further preferred embodiment, the value of a marker of the present invention is determined from the polar phase or from the lipophilic phase. The skilled person knows how to adjust parameters to ensure that a given marker is comprised in either the polar phase or the lipophilic phase. In a further preferred embodiment, the values of at least two, or at least three markers of the present invention are determined from the polar phase or from the lipophilic phase. In a preferred embodiment, the values of all markers of a panel are determined from the polar phase. In another preferred embodiment, the values of all markers of a panel are determined from the lipophilic phase.

In a preferred embodiment, at least one, at least two, or at least three markers of a panel comprised in a sample are derivatized as specified elsewhere herein and in the Examples. In a further preferred embodiment, all markers of a panel comprised in a sample are derivatized as specified elsewhere herein and in the Examples. In a preferred embodiment, the markers comprised in the polar phase are derivatized by methoxymation and silylation, preferably trimethylsilylation. In a further preferred embodiment, the markers comprised in the lipophilic phase are derivatized by transmethylation, methoxymation and silylation, preferably trimethylsilylation.

In a further preferred embodiment, at least one, at least two, or at least three markers of a panel comprised in a sample are not derivatized. In a further preferred embodiment, no marker of a panel comprised in a sample is derivatized.

The sample referred to in accordance with the present invention is, preferably, derived from a subject. The term “subject”, as used herein, relates to an animal and, preferably, to a mammal. More preferably, the subject is a farm, laboratory or companion animal, including, e.g. preferably, a mouse, a rat, a goat, a sheep, a pig, a horse, a donkey, a dog, a cat, a guinea pig, or a primate. Most preferably, the subject is a human. The subject, preferably, is suspected to suffer from a disease or medical condition, or not, or is at risk for developing a disease or medical condition, or not.

The term “value” is understood by the skilled person and relates to any measured or calculated parameter based on measuring a concentration of at least one of the biomarkers of the present invention. Preferably, the value is an absolute or relative concentration of a biomarker or a ratio of the concentrations of at least two biomarkers, preferably an intra-sample ratio of at least two biomarkers.

The term “determining the value”, accordingly, preferably relates to determining the value of a marker of the present invention. Preferably, determining the value is determining a calculated value derived from at least one concentration value of a biomarker, or determining the value is determining the amount of a biomarker as specified herein below. The term “determining the amount”, as used herein, relates to determining at least one characteristic feature of a biomarker to be determined by the method of the present invention in the sample. Characteristic features in accordance with the present invention are features which characterize the physical and/or chemical properties, including biochemical properties, of a marker. Such properties include, e.g., molecular weight, viscosity, density, electrical charge, spin, optical activity, color, fluorescence, chemoluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a biomarker by standard operations, e.g., mathematical calculations such as multiplication, division or logarithmic calculus. Most preferably, the at least one characteristic feature allows the determination and/or chemical identification of the said at least one marker and its amount. Accordingly, the characteristic value, preferably, also comprises information relating to the abundance of the biomarker from which the characteristic value is derived. For example, a characteristic value of a biomarker may be a peak in a mass spectrum. Such a peak contains characteristic information of the biomarker, i.e. the m/z information, as well as an intensity value being related to the abundance of the said biomarker (i.e. its amount) in the sample.

In a preferred embodiment, the value for the characteristic feature can also be a calculated value or a combined value such as score of a classification algorithm like “elastic net” as set forth elsewhere herein. In a preferred embodiment, it is envisaged to calculate a score, in particular a single score, based on the amounts of the markers of the method of the present invention, and to compare this score to a reference score. Preferably, the combined value (“score”) is based on the amounts of the markers in the sample from the blood product. For example, if the amounts of the biomarkers of panel 3_a are determined, the calculated score is based on the amounts of Ornithine, Glycerol-3-phosphate, and Glycerate in the sample. In a preferred embodiment, the calculated score combines information on the amounts of the markers. Preferably, in the score, the markers are, weighted in accordance with their contribution to the establishment of the result. Based on the combination of markers applied in the method of the invention, the weight of an individual biomarker may be different. The score can, preferably, be regarded as a classifier parameter for assessing the quality of a blood product sample. More preferably, it enables the assessment based on a single score based on the comparison with a reference score. The reference score is preferably a value, in particular a cut-off value which allows for differentiating between sufficient quality and insufficient quality of a blood product. Preferably, the reference is a single value; thus, the operating person does not have to interpret the entire information on the amounts of the individual markers. Preferably, using a scoring system as described herein, advantageously, values of different dimensions or units for the biomarkers may be used since the values will be mathematically transformed into the score. Thus, in a preferred embodiment of the present invention, the comparison of the amounts of the markers to a reference as set forth in step b) of the method of the present invention encompasses step b1) of calculating a score based on the determined values of the markers as referred to in step a), and step b2) of comparing the, thus, calculated score to a reference score. More preferably, a logistic regression method is used for calculating the score and, most preferably, said logistic regression method comprises elastic net regularization.

In a further preferred embodiment, the amount of each of the markers is compared to a corresponding reference, wherein the result of this comparison is used for the calculation of a combined value (score), in particular a single score, and wherein said score is compared to a reference score as specified elsewhere herein.

In a preferred embodiment, a score is calculated based on a suitable scoring algorithm. Said scoring algorithm, preferably, shall allow for differentiating whether a blood product is of sufficient quality, or not, based on the values of the markers determined. Preferably, said scoring algorithm has been previously determined by comparing the information regarding the amounts of the individual markers in samples of known sufficient quality and from samples of known insufficient quality. Accordingly, step b) if the method of the present invention may also comprise step b0) of determining or implementing a scoring algorithm. Preferably, this step is carried out prior steps b1) and b2). A suitable scoring algorithm can be determined with the markers of the present invention by the skilled person without further ado. E.g., the scoring algorithm may be a mathematical function that uses information regarding the amounts of the markers in a number of samples of sufficient and insufficient quality. Methods for determining a scoring algorithm are well known in the art and including Significance Analysis of Microarrays, Tree Harvesting, CART, MARS, Self Organizing Maps, Frequent Item Set, Bayesian networks, Prediction Analysis of Microarray (PAM), SMO, Simple Logistic Regression, Logistic Regression, Multilayer Perceptron, Bayes Net, Naïve Bayes, Naïve Bayes Simple, Naïve Bayes Up, IB1, Ibk, Kstar, LWL, AdaBoost, ClassViaRegression, Decorate, Multiclass Classifier, Random Committee, j48, LMT, NBTree, Part, Random Forest, Ordinal Classifier, Sparse Linear Programming (SPLP), Sparse Logistic Regression (SPLR), Elastic net, Support Vector Machine, Prediction of Residual Error Sum of Squares (PRESS), Penalized Logistic Regression, Mutual Information. Typically, a classification algorithm such as those implementing the elastic net method may be used for scoring (Zou 2005, Journal of the Royal Statistical Society, Series B: 301-320, Friedman 2010, J. Stat. Sotw. 33). Thus, the score for a blood product can be, preferably, calculated with a logistic regression model fitted, e.g., by using the elastic net algorithm such as implemented in the R package glmnet.

In a preferred embodiment, a reference combined value (“reference score”) is obtained from a sample from blood products of known insufficient quality. In such a case, a score in the sample being essentially identical to the reference score is indicative for insufficient quality. Moreover, the reference score, also preferably, could be from a sample from blood products of known sufficient quality. In such a case, a score in the test sample being altered, with respect to the reference score is indicative for insufficient quality. Alternatively, a score in the sample being essentially identical to said reference score is indicative sufficient quality.

In a preferred embodiment of the present invention (e.g. of the methods, devices, uses etc.), the reference score is cut-off value, preferably a single cut-off value. Preferably, said value allows for allocating the blood product either into a group of blood product of sufficient quality or into a group of blood products of insufficient quality. In another preferred embodiment of the present invention (e.g. of the methods, devices, uses etc.), the reference score is a reference score range. In this context, a reference score range indicative for sufficient quality, a reference score range indicative for insufficient quality, or two reference score ranges (i.e. a reference score range indicative for sufficient quality and a reference score range indicative for insufficient quality) can be applied.

As discussed before, each biomarker comprised by a sample may be, preferably, determined in accordance with the present invention quantitatively or semi-quantitatively. For quantitative determination, either the absolute or precise amount of the biomarker will be determined or the relative amount of the biomarker will be determined based on the value determined for the characteristic feature(s) referred to herein above. The relative amount may be determined in a case were the precise amount of a biomarker can or shall not be determined. In said case, it can be determined whether the amount in which the biomarker is present is increased or diminished with respect to a second sample comprising said biomarker in a second amount. In a preferred embodiment said second sample comprising said biomarker shall be a calculated reference as specified elsewhere herein. Quantitatively analyzing a biomarker, thus, also includes what is sometimes referred to as semi-quantitative analysis of a biomarker.

Moreover, determining as used in the method of the present invention, preferably, includes using a compound separation step prior to the analysis step referred to before. Preferably, said compound separation step yields a time and/or space resolved separation of the metabolites comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or GC are chromatographic techniques to be envisaged by the method of the present invention. Suitable devices for such determination of biomarkers are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotron-resonance mass spectrometry (FT-ICR-MS), capillary electrophoresis mass spectrometry (GE-MS), high-performance liquid chromatography coupled mass spectrometry (HPLC-MS), ultra high-performance liquid chromatography mass spectrometry (UPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Said techniques are disclosed in, e.g., Nissen 1995, Journal of Chromatography A, 703: 37-57, U.S. Pat. No. 4,540,884 or U.S. Pat. No. 5,397,894, the disclosure content of which is hereby incorporated by reference. More preferably, mass spectrometry as used herein encompasses quadrupole MS. Most preferably, said quadrupole MS is carried out as follows: a) selection of a mass/charge quotient (m/z) of an ion created by ionization in a first analytical quadrupole of the mass spectrometer, b) fragmentation of the ion selected in step a) by applying an acceleration voltage in an additional subsequent quadrupole which is filled with a collision gas and acts as a collision chamber, c) selection of a mass/charge quotient of an ion created by the fragmentation process in step b) in an additional subsequent quadrupole, whereby steps a) to c) of the method are carried out at least once and analysis of the mass/charge quotient of all the ions present in the mixture of substances as a result of the ionization process, whereby the quadrupole is filled with collision gas but no acceleration voltage is applied during the analysis. Details on said most preferred mass spectrometry to be used in accordance with the present invention can be found in WO2003/073464. As an alternative or in addition to mass spectrometry techniques, the following techniques may be used for compound determination: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionization detection (FID). These techniques are well known to the person skilled in the art and can be applied without further ado.

More preferably, mass spectrometry is LC-MS and/or GC-MS, i.e. mass spectrometry being operatively linked to a prior chromatographic separation step. Liquid chromatography as used herein refers to all techniques which allow for separation of compounds (i.e. metabolites) in liquid or supercritical phase. Liquid chromatography is characterized in that compounds in a mobile phase are passed through the stationary phase. When compounds pass through the stationary phase at different rates they become separated in time since each individual compound has its specific retention time (i.e. the time which is required by the compound to pass through the system). Liquid chromatography as used herein also includes HPLC. Devices for liquid chromatography are commercially available, e.g. from Agilent Technologies, USA. Gas chromatography as applied in accordance with the present invention, in principle, operates comparable to liquid chromatography. However, rather than having the compounds (i.e. metabolites) in a liquid mobile phase which is passed through the stationary phase, the compounds will be present in a gaseous volume. The compounds pass the column which may contain solid support materials as stationary phase or the walls of which may serve as or are coated with the stationary phase. Again, each compound has a specific time which is required for passing through the column. Moreover, in the case of gas chromatography it is preferably envisaged that the compounds are derivatized prior to gas chromatography. Suitable techniques for derivatization are well known in the art. Preferably, derivatization in accordance with the present invention relates to methoxymation and silylation, more preferably trimethylsilylation of, preferably, polar compounds and transmethylation, methoxymation and silylation, more preferably trimethylsilylation of, preferably, non-polar (i.e. lipophilic) compounds. Moreover, the at least one biomarker can also be determined by a specific chemical or biological assay. Said assay shall comprise means which allow to specifically detect the at least one biomarker in the sample. Preferably, said means are capable of specifically recognizing the chemical structure of the biomarker or are capable of specifically identifying the biomarker based on its capability to react with other compounds or its capability to elicit a response in a biological read out system (e.g., induction of a reporter gene). Means which are capable of specifically recognizing the chemical structure of a biomarker are, preferably, antibodies or other proteins which specifically interact with chemical structures, such as receptors or enzymes. Specific antibodies, for instance, may be obtained using the biomarker as antigen by methods well known in the art. Antibodies as referred to herein include both polyclonal and monoclonal antibodies, as well as fragments thereof, such as Fv, Fab and F(ab)2 fragments that are capable of binding the antigen or hapten. The present invention also includes humanized hybrid antibodies wherein amino acid sequences of a non-human donor antibody exhibiting a desired antigen-specificity are combined with sequences of a human acceptor antibody. Moreover, encompassed are single chain antibodies. The donor sequences will usually include at least the antigen-binding amino acid residues of the donor but may comprise other structurally and/or functionally relevant amino acid residues of the donor antibody as well. Such hybrids can be prepared by several methods well known in the art. Suitable proteins which are capable of specifically recognizing the biomarker are, preferably, enzymes which are involved in the metabolic conversion of the said biomarker. Said enzymes may either use the biomarker as a substrate or may convert a substrate into the biomarker. Moreover, said antibodies may be used as a basis to generate oligopeptides which specifically recognize the biomarker. These oligopeptides shall, for example, comprise the enzyme's binding domains or pockets for the said biomarker. Suitable antibody and/or enzyme based assays may be RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA) or solid phase immune tests. Moreover, the biomarker may also be determined based on its capability to react with other compounds, i.e. by a specific chemical reaction. Further, the biomarker may be determined in a sample due to its capability to elicit a response in a biological read out system. The biological response shall be detected as read out indicating the presence and/or the amount of the biomarker comprised by the sample. The biological response may be, e.g., the induction of gene expression or a phenotypic response of a cell or an organism. In a preferred embodiment, the determination of the least one biomarker is a quantitative process, e.g., allowing also the determination of the amount of the at least one biomarker in the sample.

The term “reference”, as used herein, refers to values or ranges of values of characteristic features of a marker of the present invention in a sample or in a plurality of samples of known quality. Preferably, a reference is a threshold value (e.g., an amount or ratio of amounts) for a marker whereby said threshold divides the range of possible values for the characteristic features into a first and a second part. One of these parts is associated with insufficient quality while the other is associated with sufficient quality. The threshold value itself may also be associated with either sufficient or insufficient quality. In case the threshold is associated with insufficient quality, values found in a sample to be investigated which are, therefore, essentially identical to the threshold or which fall into the part associated with insufficient quality indicate insufficient quality of the sample. In case the threshold is associated with sufficient quality, values found in a sample to be investigated which are essentially identical to the threshold or which fall into the part associated with sufficient quality indicate sufficient quality of the sample. Accordingly, preferably, the threshold value is a cut-off value. As detailed herein above, in case assessing relates to classifying a sample into one of two classes, e.g., preferably, acceptable quality or inacceptable quality, the reference may, preferably, be a threshold or cut-off value. It will be understood by the skilled person that, in case, classification into more than two classes is performed, more than one reference value may be relevant in said classification, e.g., preferably, two reference values may be used to define the boundaries between three classes.

Preferably, the reference values, e.g., preferably, values for at least one characteristic feature of the at least one marker or ratios thereof, will be stored in a suitable data storage medium such as a database and are, thus, also available for future assessments. As will be understood by the skilled person, an unexpectedly high deviation from the reference value, e.g. preferably, more than tenfold, may also be caused by a systematic error, which can be, as non-limiting examples, faulty dilution of a sample or device malfunction; the skilled person knows that results should be counterchecked in such case.

In accordance with the aforementioned method of the present invention, a reference is, preferably, a reference obtained from a sample or plurality of samples (i.e., preferably, more than 1, 2, 3, 4, 5, 10, 50 or 100 samples) of known quality. How to calculate a suitable reference value, preferably, the average or median, is well known in the art. Preferably, the reference is derived from a sample or plurality of samples known to be of insufficient quality. In such a case, a value for a marker found in the sample being essentially identical is indicative for insufficient quality while a value for the marker found in the sample being different is indicative for sufficient quality. Preferably, in such case, a sample is classified as having insufficient quality if at least x marker(s) of a panel are essentially identical to said reference, with x being selected from the list consisting of 1, 2, . . . , (n−1) and n=Number of markers in said panel; more preferably, a sample is classified as having insufficient quality if all markers of a panel are essentially identical to said reference.

Also preferably, the said reference is derived from a sample or plurality of samples known to be of sufficient quality. More preferably, in such a case a value of a marker in the sample being essentially identical to the said reference is indicative for sufficient quality, while an amount which differs therefrom is indicative for insufficient quality. Preferably, a change in the direction indicated in Table 1, Table 2, or Table 2a is indicative for insufficient quality. Preferably, in such case, a sample is classified as having insufficient quality if at least x marker(s) of a panel is different from said reference, with x being selected from the list consisting of 1, 2, . . . , (n−1) and n=Number of markers in said panel; more preferably, a sample is classified as having insufficient quality if all markers of a panel are different from said reference. Preferably, in case the sample is a blood product sample, a sample known to be of sufficient quality is a sample obtained according to a standard protocol as specified elsewhere herein.

The value for the at least one marker of the test sample and the reference value are essentially identical, if the values for the characteristic features and, in the case of quantitative determination, the intensity values, or the values calculated therefrom are essentially identical. Essentially identical means that the difference between two values is, preferably, not significant and shall be characterized in that the values are within at least the interval between 1st and 99th percentile, 5th and 95th percentile, 10th and 90th percentile, 20th and 80th percentile, 30th and 70th percentile, 40th and 60th percentile of the reference value, preferably, the 50th, 60th, 70th, 80th, 90th or 95th percentile of the reference value. Statistical tests for determining whether two amounts are essentially identical are well known in the art and are also described elsewhere herein.

An observed difference for two values, on the other hand, shall preferably be statistically significant. A difference in the value is, preferably, significant outside of the interval between 45th and 55th percentile, 40th and 60th percentile, 30th and 70th percentile, 20th and 80th percentile, 10th and 90th percentile, 5th and 95th percentile, 1st and 99th percentile of the reference value. In the Tables of this specification, a preferred relative change for the biomarkers is indicated as “up” for an increase and “down” for a decrease in column “direction”.

The term “corresponding reference” is understood by the skilled person and relates to a value obtained for the same marker from a different sample, preferably in a reference sample. It is understood by the skilled person that, e.g., preferably, an intra-sample ratio of two biomarkers is compared to a reference intra-sample ratio of the same biomarkers and that a relative concentration of a biomarker is compared to a reference relative concentration of the same biomarker, and the like.

The term “comparing” refers to determining whether the determined value of a marker is essentially identical to a reference or differs therefrom. Preferably, a value for a marker is deemed to differ from a reference if the observed difference is statistically significant which can be determined by statistical techniques referred to elsewhere in this description. If the difference is not statistically significant, the biomarker value and the reference are essentially identical. Based on the comparison referred to above, the quality of a sample can be assessed, i.e. it can be assessed whether the sample is of sufficient quality, or not. The comparison is, preferably, assisted by automation. For example, a suitable computer program comprising algorithms for the comparison of two different data sets (e.g., data sets comprising the values of the characteristic feature(s)) may be used. Such computer programs and algorithms are well known in the art. Notwithstanding the above, a comparison can also be carried out manually.

Advantageously, it has been found in the study underlying the present invention that the specific combinations of markers (panels) allow to sensitively detect whether a sample was exposed to one of the confounding factors as specified herein. Surprisingly, statistical analysis showed that not the markers showing the highest AUC values when used as single markers are most suitable for this determination. Apparently, markers being of lower indicative value as single markers show synergistic effects with other markers, leading to a surprisingly potent method of analyzing quality of a sample. The predictive value of the panels of Table 1 could be further increased by including further markers, leading to the optimized panels of Table 2 and Table 2a.

The definitions made above apply mutatis mutandis to the following. Additional definitions and explanations made further below also apply for all embodiments described in this specification mutatis mutandis.
The present invention further relates to a device for assessing the quality of a blood product sample comprising:
   a) an analyzing unit for said sample, comprising at least one detector for at least the markers of at least one panel of Table 1, said at least one detector determining the amounts of said markers in said sample; and, operatively linked thereto,
   b) an evaluation unit comprising a data processing unit and a database, said database comprising stored corresponding reference values and said data processing unit optionally having tangibly embedded an algorithm calculating an intra-sample ratio of two biomarkers and comparing the values of the markers determined by the analyzing unit or the values calculated by the evaluation unit to said stored reference values and generating an output information based on which assessment of the quality is established.

A “device”, as the term is used herein, shall comprise at least the aforementioned units. The units of the device are operatively linked to each other. How to link the means in an operating manner will depend on the type of units included into the device. For example, where the detector allows for automatic qualitative or quantitative determination of the biomarker, the data obtained by said automatically operating analyzing unit can be processed by, e.g., a computer program in order to facilitate the assessment in the evaluation unit. Preferably, the units are comprised by a single device in such a case. Preferably, the device includes an analyzing unit for the biomarker and a computer or data processing device as an evaluation unit for processing the resulting data for the assessment and for establishing the output information. Preferably, the analyzing unit comprises at least one detector for at least the markers of a panel according to the present invention, or, more preferably, (i) for at least the markers glycerol-3-phosphate, glycerate, and ornithine; (ii) for at least glycerol-3-phosphate, glycerate, and hypoxanthine; (iii) for at least glycerol-3-phosphate, ornithine, and hypoxanthine; or (iv) for at least glycerate, ornithine, and hypoxanthine; or in particular for at least the markers glycerol-3-phosphate, glycerate, and ornithine; said at least one detector determining the amounts of said markers in said sample. Preferred devices are those which can be applied without the particular knowledge of a specialized clinician, e.g., electronic devices which merely require loading with a sample. The output information of the device, preferably, is a numerical value which allows drawing conclusions on the quality of the sample and, thus, is an aid for the reliability of a diagnosis or for troubleshooting. In a preferred embodiment, the analyzing unit of the device comprises at least one detector for at least the markers of panel 3_a, 13_a, 15_a, 16_a, 1_a, 1_b, 10_a, 11_a, 12_a, 13_b, 14_a, 14_b, 17_b, 18_b, 19_a, 19_b, 2_b, 20_b, 3_b, 4_a, 5_a, 5_b, 6_a, 7_a, 7_b, 8_b, 1, 2, 3, 4, 5, 6, 7, 8, 10, 11, 12, 13, 14, 15, 16, 17, 18, 19, 20, 3_m, 6_m, 10_m, 15_m, 16_m, 17_m, 18_m, 19_m, or 20_m.

Preferred references to be used as stored references in accordance with the device of the present invention are values for the markers to be analyzed or values derived therefrom which are derived from a sample or plurality of samples of insufficient quality. In such a case, the algorithm tangibly embedded, preferably, compares the determined values for the markers with the references, wherein identical or essentially identical values shall be indicative for a sample of insufficient quality, while values which differ, preferably, which show a change in the direction opposite to the direction indicated in table 1, 2, 2a, or 3, indicate a sample of sufficient quality.

Alternatively, other preferred references to be used as stored references in accordance with the device of the present invention are values for the markers to be analyzed or values derived therefrom which are derived from a sample or plurality of samples of sufficient quality. In such a case, the algorithm tangibly embedded, preferably, compares the determined values for the markers with the references, wherein identical or essentially identical values shall be indicative for a sample of sufficient quality, while values which differ indicates a sample of insufficient quality.

The units of the device, also preferably, can be implemented into a system comprising several devices which are operatively linked to each other. Depending on the units to be used for the system of the present invention, said means may be functionally linked by connecting each means with the other by means which allow data transport in between said means, e.g., glass fiber cables, and other cables for high throughput data transport. Nevertheless, wireless data transfer between the means is also envisaged by the present invention, e.g., via LAN (Wireless LAN, W-LAN). A preferred system comprises means for determining biomarkers. Means for determining biomarkers as used herein encompass means for separating biomarkers, such as chromatographic devices, and means for metabolite determination, such as mass spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS and/or GC-MS are used in the system of the present invention as described in detail elsewhere in the specification. Further comprised shall be means for comparing and/or analyzing the results obtained from the means for determination of biomarkers. The means for comparing and/or analyzing the results may comprise at least one databases and an implemented computer program for comparison of the values measured with corresponding references. Preferred embodiments of the aforementioned systems and devices are also described in detail below.

Furthermore, the present invention relates to a data collection comprising characteristic values of at least the markers of at least one panel of Table 1, Table 2, or Table 2a, being indicative for sufficient or insufficient quality of a sample.

The term “data collection” refers to a collection of data which may be physically and/or logically grouped together. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for a sample quality as set forth above (e.g. a query search). Thus, if an identical or similar data set can be identified in the data collection, the test data set will be associated with the said quality. Consequently, the information obtained from the data collection can be used, e.g., as a reference for the methods of the present invention described above. More preferably, the data collection comprises characteristic values of all biomarkers comprised by any one of the groups recited above.

In light of the foregoing, the present invention encompasses a data storage medium comprising the aforementioned data collection.

The term “data storage medium” as used herein encompasses data storage media which are based on single physical entities such as a CD, a CD-ROM, a hard disk, optical storage media, or a diskette. Moreover, the term further includes data storage media consisting of physically separated entities which are operatively linked to each other in a manner as to provide the aforementioned data collection, preferably, in a suitable way for a query search.

The present invention also relates to an analysis system comprising:
(a) means for comparing characteristic values of markers of a sample operatively linked to
(b) a data storage medium according to the present invention.

The term “analysis system”, as used herein, relates to different means which are operatively linked to each other. Said means may be implemented in a single device or may be physically separated devices which are operatively linked to each other. The means for comparing characteristic values of markers, preferably, based on an algorithm or score for comparison as mentioned before. The data storage medium, preferably, comprises the aforementioned data collection or database, wherein each of the stored data sets being indicative for a sample quality referred to above. Thus, the analysis system of the present invention allows identifying whether a test data set is comprised by the data collection stored in the data storage medium. Consequently, the methods of the present invention can be implemented by the analysis system of the present invention.

In a preferred embodiment of the analysis system, means for determining characteristic values of biomarkers of a sample are comprised. The term “means for determining characteristic values of biomarkers” preferably relates to the aforementioned devices for the determination of metabolites such as mass spectrometry devices, NMR devices or devices for carrying out chemical or biological assays for the biomarkers.

The present invention also relates to a use of at least the markers of at least one panel of Table 1, or of a detection agent or detection reagents therefor, for assessing the quality of a blood product sample. Preferably, said panel comprises (i) glycerol-3-phosphate, glycerate, and ornithine; (ii) glycerol-3-phosphate, glycerate, and hypoxanthine; (iii) glycerol-3-phosphate, ornithine, and hypoxanthine; (iv) glycerate, ornithine, and hypoxanthine; or (v) glutamine, glycerol-3-phosphate, glutamate, and hypoxanthine; in particular said panel comprises glycerol-3-phosphate, glycerate, and ornithine or said panel comprises glutamine, glycerol-3-phosphate, glutamate, and hypoxanthine. In a preferred embodiment, said at least one panel is panel 3_a, 13_a, 15_a, 16_a, 1_a, 1_b, 10_a, 11_a, 12_a, 13_b, 14_a, 14_b, 17_b, 18_b, 19_a, 19_b, 2_b, 20_b, 3_b, 4_a, 5_a, 5_b, 6_a, 7_a, 7_b, 8_b, 1, 2, 3, 4, 5, 6, 7, 8, 10, 11, 12, 13, 14, 15, 16, 17, 18, 19, 20, 3_m, 6_m, 10_m, 15_m, 16_m, 17_m, 18_m, 19_m, or 20_m.

Moreover, the present invention relates to a kit for assessing the quality of a blood product sample comprising at least one detection agent for at least the markers of at least one panel of Table 1, and/or references for the said markers, comprised in a housing.

The term “kit”, as used herein, refers to a collection of the aforementioned components, preferably, provided separately or within a single container. The container also comprises instructions for carrying out the method of the present invention. These instructions may be in the form of a manual. Preferably, instructions are directions how to establish quality assessment, most preferably including instructions enabling the user to establish quality assessment. Said instructions, preferably, are provided by a computer program code which is capable of carrying out the comparisons referred to in the methods of the present invention and to establish a quality assessment of a sample when implemented on a computer or a data processing device. The computer program code may be provided on a data storage medium or device such as an optical storage medium (e.g., a Compact Disc) or directly on a computer or data processing device. In another embodiment, the container does not comprise instructions for carrying out the method of the present invention; thus, in a preferred embodiment, the kit is a collection of the aforementioned components, preferably, provided separately or within a single container. Further, the kit shall, preferably, comprise at least one standard for a reference per biomarker as defined herein above, i.e. a solution with a pre-defined amount for the at least one biomarker representing a reference amount. Such a standard may represent, e.g., the amount of a biomarker from a sample or plurality of samples of sufficient or insufficient quality. In a preferred embodiment, the kit comprises at least one detection agent for at least the markers of panel 3_a, 13_a, 15_a, 16_a, 1_a, 1_b, 10_a, 11_a, 12_a, 13_b, 14_a, 14_b, 17_b, 18_b, 19_a, 19_b, 2_b, 20_b, 3_b, 4_a, 5_a, 5_b, 6_a, 7_a, 7_b, 8_b, 1, 2, 3, 4, 5, 6, 7, 8, 10, 11, 12, 13, 14, 15, 16, 17, 18, 19, 20, 3_m, 6_m, 10_m, 15_m, 16_m, 17_m, 18_m, 19_m, or 20_m.

How detection agents can be manufactured based on a biomarker is well known to those skilled in the art. For example, antibodies or aptamers which specifically bind to the at least one biomarker can be produced. Similarly, the biomarker itself may be used in a kit as a reference, e.g., within complexes or in modified or derivatized form, e.g., when analyzed by GCMS. It is understood by the skilled person that in case the marker is a calculated value, the detection agent, preferably, is a combination of detection agents for determining the biomarkers used for calculating the value of said marker.

Moreover, the present invention relates to a method of providing a collection of blood products or of blood product samples of sufficient quality, comprising
a) providing a pool of blood products or of blood product samples,
b) performing the steps of the method for assessing the quality of a blood product sample of the present invention on a sample of each member of said pool of blood products or on each member of said pool of blood product samples,
c) discarding a blood product or blood product sample in case insufficient quality is assessed, and/or excluding a blood product or blood product sample from further use in case insufficient quality is assessed; thereby providing a collection of blood products or of blood product samples of sufficient quality.

The method of providing a collection of blood products of sufficient quality of the present invention, preferably, is an in vitro method. Moreover, it may comprise steps in addition to those explicitly mentioned above. Moreover, one or more of said steps may be performed by automated equipment.

All references cited in this specification are herewith incorporated by reference with respect to their entire disclosure content and the disclosure content specifically mentioned in this specification.

In view of the above, the following embodiments are preferred:

### EMBODIMENT 1

A method for assessing the quality of a blood product sample, comprising:
a) determining in said sample the values of the markers of at least one panel of Table 1;
b) comparing the values determined in step a) with corresponding references, and,
c) assessing the quality of said blood product sample.

### EMBODIMENT 2

The method of embodiment 1, wherein in step a) the amounts of the markers (i) glycerol-3-phosphate, glycerate, and ornithine; (ii) glycerol-3-phosphate, glycerate, and hypoxanthine; (iii) glycerol-3-phosphate, ornithine, and hypoxanthine; or (iv) glycerate, ornithine, and hypoxanthine are determined.

### EMBODIMENT 3

The method of embodiment 1, wherein in step a) the values of the markers of at least one panel of Table 2 are determined.

### EMBODIMENT 4

The method of any one of embodiments 1 to 3, wherein in step a) the values of the markers of at least one of panels 3, 13, 15, 18, 19, or 20 of Table 2 are determined.

### EMBODIMENT 5

The method of any one of embodiments 1 to 4, wherein assessing the quality of a blood product sample is ensuring that said blood product sample has not been affected by any of the confounding factors (i) prolonged time between phlebotomy and separation of plasma from blood cells, (ii) increased temperature between phlebotomy and separation of plasma from blood cells, (iii) prolonged time of storage of plasma, and (iv) increased temperature during storage of plasma.

### EMBODIMENT 6

The method of any one of embodiments 1 to 5, wherein in case a blood product sample of insufficient quality is identified, said method comprises the further step of differentiating whether said sample has been compromised by confounding factors related to blood processing or by confounding factors related to plasma processing.

### EMBODIMENT 7

The method of embodiment 6, wherein said confounding factors related to blood processing are (i) prolonged time between phlebotomy and separation of plasma from blood cells, (ii) increased temperature between phlebotomy and separation of plasma from blood cells.

### EMBODIMENT 8

The method of embodiment 6 or 7, wherein said confounding factors related to plasma processing are (i) prolonged time of storage of plasma, and (ii) increased temperature during storage of plasma.

### EMBODIMENT 9

The method of any one of embodiments 1 to 8, wherein in step a) additionally the amounts of the markers ethylenediaminetetraacetic acid (EDTA), citrate, and aspartate are determined, and wherein in step b) the amounts of said additional markers are compared to corresponding references.

### EMBODIMENT 10

The method of embodiment 9, wherein assessing the quality of a blood product sample further comprises differentiating whether said sample has been compromised by factors related to collection tube selection.

### EMBODIMENT 11

The method of any one of embodiments 1 to 10, wherein said reference values are obtained by determining the values of the markers in a sample processed according to the conditions: (i) Blood processing within 60 min between blood draw and centrifugation at a temperature between 18° C. and 22° C. and (ii) storage of plasma at a temperature of less than 5° C. for less than 30 min, and (iii) storage of plasma at a temperature of less than −80° C. for less than 1 year.

### EMBODIMENT 12

The method of any one of embodiments 1 to 11, wherein said blood product sample is a blood sample or a plasma sample.

### EMBODIMENT 13

The method of any one of embodiments 1 to 12, wherein determining the values of said markers comprises reacting at least one, preferably all, of said markers with an enzyme or enzymes.

### EMBODIMENT 14

The method of any one of embodiments 1 to 12, wherein determining the values of said markers comprises a mass spectrometry (MS) method.

### EMBODIMENT 15

The method of any one of embodiments 1 to 12 and 14, wherein determining the values of said markers comprises a combination of solid phase extraction (SPE) with liquid chromatography (LC) and mass spectrometry (MS), preferably SPE-LC-MS/MS or SPE-Ultra Performance Liquid Chromatography (UPLC)-MS/MS.

### EMBODIMENT 16

The method of any one of embodiments 1 to 12 and 14, wherein determining the values of said markers comprises a combination of LC with mass spectrometry, preferably LC-MS/MS.

### EMBODIMENT 17

The method of any one of embodiments 1 to 12 and 14, wherein determining the values of said markers comprises a combination of gas chromatography (GC) with mass spectrometry (MS), preferably GC-MS or GC-MS/MS.

### EMBODIMENT 18

The method of any one of embodiments 1 to 17, further comprising the step of determining an internal standard for at least one of said markers, preferably for all of said markers.

### EMBODIMENT 19

The method of any one of embodiments 1 to 18, further comprising the step of determining an external standard for at least one of said markers, preferably for all of said markers.

### EMBODIMENT 20

The method of any one of embodiments 1 to 19, wherein said determining the value of a marker is determining the amount of said marker or is determining a calculated value derived from at least one concentration value of a marker, preferably, a ratio of the concentrations of at least two biomarkers.

### EMBODIMENT 21

The method of any one of embodiments 1 to 20, wherein individual numerical values of said markers are translated into a combined value by using a multivariate model, preferably, a logistic regression model.

### EMBODIMENT 22

The method of any one of embodiments 1 to 21, wherein said step b) comprises the steps of
b1) calculating a combined value based on the determined values of said markers as referred to in step a), wherein, preferably, in said calculating a combined value the markers are weighted due to their importance; and
b2) comparing the, thus, calculated combined value to a reference combined value.

### EMBODIMENT 23

The method of any one of embodiments 1 to 21, wherein said step b) comprises the steps of
b1) comparing the values determined in step a) with corresponding references, and calculating a combined value based on said comparison, wherein, preferably, in said calculating a combined value the markers are weighted due to their importance; and
b2) comparing the, thus, calculated combined value to a reference combined value.

### EMBODIMENT 24

The method of any one of embodiments 1 to 23, wherein said assessing the quality of a blood product sample is establishing a numerical quality assessment of a sample and wherein said values of said markers are categorized by comparison to pre-defined cut-offs.

### EMBODIMENT 25

The method of embodiment 24, wherein said markers categorized by comparison to pre-defined cut-offs are combined into a single value which is then scaled, and wherein, preferably, in said scaling the markers are weighted due to their importance.

### EMBODIMENT 26

The method of any one of embodiments 1 to 25, comprising determining the values of the markers of at least panel 3_a, 13_a, 15_a, 16_a, 1_a, 1_b, 10_a, 11_a, 12_a, 13_b, 14_a, 14_b, 17_b, 18_b, 19_a, 19_b, 2_b, 20_b, 3_b, 4_a, 5_a, 5_b, 6_a, 7_a, 7_b, 8_b, 1, 2, 3, 4, 5, 6, 7, 8, 10, 11, 12, 13, 14, 15, 16, 17, 18, 19, 20, 3_m, 6_m, 10_m, 15_m, 16_m, 17_m, 18_m, 19_m, or 20_m.

### EMBODIMENT 27

A device for assessing the quality of a blood product sample comprising:
a) an analyzing unit for said sample, comprising at least one detector for at least the markers of at least one panel of Table 1, Table 2, or Table 2a, said at least one detector determining the amounts of said markers in said sample; and, operatively linked thereto,
b) an evaluation unit comprising a data processing unit and a database, said data base comprising stored corresponding reference values and said data processing unit optionally having tangibly embedded an algorithm calculating an intra-sample ratio of two biomarkers and comparing the values of the markers determined by the analyzing unit or the values calculated by the evaluation unit to said stored reference values and generating an output information based on which assessment of the quality is established.

### EMBODIMENT 28

The device of embodiment 27, wherein said analyzing unit comprises at least one detector (i) for at least the markers glycerol-3-phosphate, glycerate, and ornithine; (ii) for at least the markers glycerol-3-phosphate, glycerate, and hypoxanthine; (iii) for at least the markers glycerol-3-phosphate, ornithine, and hypoxanthine; (iv) for at least the markers glycerate, ornithine, and hypoxanthine; or (v) at least one detector for at least the markers glutamine, glycerol-3-phosphate, glutamate, and hypoxanthine, said at least one detector determining the amounts of said markers in said sample.

### EMBODIMENT 29

The device of embodiment 26 or 27, wherein said analyzing unit comprises at least one detector for at least the markers of panel 3_a, 13_a, 15_a, 16_a, 1_a, 1_b, 10_a, 11_a, 12_a, 13_b, 14_a, 14_b, 17_b, 18_b, 19_a, 19_b, 2_b, 20_b, 3_b, 4_a, 5_a, 5_b, 6_a, 7_a, 7_b, 8_b, 1, 2, 3, 4, 5, 6, 7, 8, 10, 11, 12, 13, 14, 15, 16, 17, 18, 19, 20, 3_m, 6_m, 10_m, 15_m, 16_m, 17_m, 18_m, 19_m, or 20_m.

### EMBODIMENT 30

Use of at least the markers of at least one panel of Table 1, Table 2, or Table 2a, or of a detection agent or detection reagents therefor, for assessing the quality of a blood product sample.

### EMBODIMENT 31

The use of embodiment 30, wherein said panels comprise (i) glycerol-3-phosphate, glycerate, and ornithine; (ii) glycerol-3-phosphate, glycerate, and hypoxanthine; (iii) glycerol-3-phosphate, ornithine, and hypoxanthine; (iv) glycerate, ornithine, and hypoxanthine; or (v) glutamine, glycerol-3-phosphate, glutamate, and hypoxanthine.

### EMBODIMENT 32

The use of embodiment 30 or 31, wherein said at least one panel is panel 3_a, 13_a, 15_a, 16_a, 1_a, 1_b, 10_a, 11_a, 12_a, 13_b, 14_a, 14_b, 17_b, 18_b, 19_a, 19_b, 2_b, 20_b, 3_b, 4_a, 5_a, 5_b, 6_a, 7_a, 7_b, 8_b, 1, 2, 3, 4, 5, 6, 7, 8, 10, 11, 12, 13, 14, 15, 16, 17, 18, 19, 20, 3_m, 6_m, 10_m, 15_m, 16_m, 17_m, 18_m, 19_m, or 20_m.

### EMBODIMENT 33

A kit for assessing the quality of a blood product sample comprising at least one detection agent for at least the markers of at least one panel of Table 1, and/or a reference for the said markers, comprised in a housing.

### EMBODIMENT 34

The kit of embodiment 33, wherein said panels comprise (i) glycerol-3-phosphate, glycerate, and ornithine; (ii) glycerol-3-phosphate, glycerate, and hypoxanthine; (iii) glycerol-3-phosphate, ornithine, and hypoxanthine; (iv) glycerate, ornithine, and hypoxanthine; or (v) glutamine, glycerol-3-phosphate, glutamate, and hypoxanthine.

### EMBODIMENT 35

The kit of embodiment 33 or 34, wherein the kit comprises at least one detection agent for at least the markers of panel 3_a, 13_a, 15_a, 16_a, 1_a, 1_b, 10_a, 11_a, 12_a, 13_b, 14_a, 14_b, 17_b, 18_b, 19_a, 19_b, 2_b, 20_b, 3_b, 4_a, 5_a, 5_b, 6_a, 7_a, 7_b, 8_b, 1, 2, 3, 4, 5, 6, 7, 8, 10, 11, 12, 13, 14, 15, 16, 17, 18, 19, 20, 3_m, 6_m, 10_m, 15_m, 16_m, 17_m, 18_m, 19_m, or 20_m.

### EMBODIMENT 36

A method of providing a collection of blood products of sufficient quality, comprising
a) providing a collection of blood products,
b) performing the steps of the method for assessing the quality of a blood product sample of any one of embodiments 1 to 26 on a sample of each member of said collection of blood products,
c) discarding a blood product in case insufficient quality is assessed, and/or excluding a blood product from further use in case insufficient quality is assessed; thereby providing a collection of blood products of sufficient quality.

### EMBODIMENT 37

A data collection comprising characteristic values of at least the markers of at least one panel of Table 1, Table 2, or Table 2a being indicative for sufficient or insufficient quality of a blood product sample.

### EMBODIMENT 38

The data collection of embodiment 37, comprising characteristic values of at least the markers of panel 3_a, 13_a, 15_a, 16_a, 1_a, 1_b, 10_a, 11_a, 12_a, 13_b, 14_a, 14_b, 17_b, 18_b, 19_a, 19_b, 2_b, 20_b, 3_b, 4_a, 5_a, 5_b, 6_a, 7_a, 7_b, 8_b, 1, 2, 3, 4, 5, 6, 7, 8, 10, 11, 12, 13, 14, 15, 16, 17, 18, 19, 20, 3_m, 6_m, 10_m, 15_m, 16_m, 17_m, 18_m, 19_m, or 20_m.

### EMBODIMENT 39

A data storage medium comprising the data collection of embodiment 37 or 38.

### EMBODIMENT 29

A system comprising:
(a) means for comparing characteristic values of the at least one biomarker of a sample operatively linked to
(b) a data storage medium according to embodiment 39.

The following Examples shall merely illustrate the invention. They shall not be construed, whatsoever, to limit the scope of the invention.

### EXAMPLES

### Example 1: Experimental Design of Creating Samples of High Quality and Low Quality with Respect to Pre-Processing of Plasma

This experiment was designed to create human plasma samples of high quality and of low quality with respect to time and temperature of plasma processing in order to identify multivariate biomarkers for quality control of blood plasma biobank specimen. An EDTA plasma pool of samples that were processed from blood to plasma within 2 h, were continuously kept at −80° C. during storage, and were not thawed and frozen again during storage was used for this experiment. The pool was divided into 1-ml-aliquots and these were incubated at temperatures of 4° C., 12° C. and 21° C. At the time points 0 h, 0.5 h, 5 h and 16 h, each 10 aliquots were frozen at −80° C. and analyzed as described in example 4 (sphingolipids were not analyzed in Example 1). Plasma samples were analyzed in randomized analytical sequence design. The raw peak data was normalized to the median of all samples per analytical sequence to account for process variability (so called “ratios”). In order to allow an experiment-comprehensive alignment of semi-quantitative data, MxPool™ (a large pool of a commercial human EDTA plasma suited for alignment of metabolic profiling studies) was analyzed with 12 replicated samples in the experiment and the ratios further normalized to the median of the MxPool™ samples, i.e. ratios from this studies are on the same level and therefore comparable to data from other projects that are normalized to other aliquots of the same MxPool™. Total quantified data from targeted methods (eicosanoids, catecholamines) remain with their absolute quantification data. Data was log 10 transformed to approach a normal distribution.

Samples processed for 0 h or 0.5 h at any temperature were considered to be of high quality; samples processed for 16 h at any temperature were considered of low quality; samples processed for 5 h at 21° C. were considered of low quality; all other samples were ignored in this approach.

### Example 2: Experimental Design of Creating Samples of High Quality and Low Quality with Respect to Processing of Blood to Plasma

This experiment was designed to create human plasma samples of high quality and of low quality with respect to pre-analytical confounders that occur during the pre-processing of blood to plasma in order to identify multivariate biomarkers for quality control of blood plasma biobank specimen.

Different groups of blood handling comprised the following procedures:
Prolonged incubation at 0° C.
Prolonged incubation at room temperature
Hemolysis

Twenty healthy volunteers (13 females, 7 males) were recruited and 64 ml of blood were withdrawn by venous puncture using a gauge-20 safety-fly blood collection system into 3 9-ml-K3EDTA monovettes followed by 1 ml into a neutral monovette (sample was discarded) followed by a 9-ml-neutral monovette followed by 3 9-ml-K3EDTA monovettes. The monovettes were gently mixed by inverting to prevent hemolysis. The K3EDTA monovettes were opened and pooled within each subject.

The blood of each subject was processed within the different groups as follows:

Prolonged Incubation at 0° C.

2×5 ml of the blood pool was incubated at 0° C. for 4 h and 6 h, respectively. After that time period, the plasma was prepared by centrifugation at 1500×g for 15 minutes in a refrigerated centrifuge. The plasma was stored at −80° C. until analysis.

Prolonged Incubation at Room Temperature

5 ml of the blood pool were incubated at room temperature for 1 h. After that time period, the plasma was prepared by centrifugation at 1500×g for 15 minutes in a refrigerated centrifuge. The plasma was stored at −80° C. until analysis.

Hemolysis

2×6 ml of the blood pool were passed through a syringe with a gauge-25 (grade 1 hemolysis) and gauge-27 needle (grade 2 hemolysis), respectively. The plasma was prepared by centrifugation at 1500×g for 15 minutes in a refrigerated centrifuge. The plasma was stored at −80° C. until analysis.

Control

The samples serving as control group were processed immediately without any delay. The remaining blood pool was centrifuged at 1500×g for 15 minutes in a refrigerated centrifuge. The upper plasma supernatant was withdrawn and mixed in a centrifugation tube. Aliquots of this plasma sample were frozen and stored at −80° C. until analysis to serve as control.

The plasma samples of this experiment were analyzed as described in example 4 in randomized analytical sequence design. Metabolite profiling provides a semi-quantitative analytical platform resulting in relative metabolite level to a defined reference group (“ratio”). To support this concept and also to allow an alignment of different analytical batches (“experiments”), two different reference sample types were run in parallel throughout the whole process. First, a project pool was generated from aliquots of all samples and measured with 4 replicates within each analytical sequence. For all semi-quantitatively analyzed metabolites, the data were normalized against the median in the pool reference samples within each analytical sequence to give pool-normalized ratios (performed for each sample per metabolite). This compensated for inter- and intra-instrumental variation. Second, MxPool™ was analyzed with 12 replicated samples in the experiment and the pool-normalized ratios further normalized to the median of the MxPool™ samples, i.e. ratios from this studies are on the same level and therefore comparable to data from other projects that are normalized to other aliquots of the same MxPool™. Total quantified data from targeted methods (eicosanoids, catecholamines) remain with their absolute quantification data.

Samples of the control group are considered to be of high quality, the other samples from this experiment are considered to be of low quality.

### Example 3: Experimental Design of Creating Samples of High Quality and Low Quality with Respect to Long-Term Storage of Plasma

This experiment was designed to create human plasma samples of high quality and of low quality with respect to long-term storage of plasma in order to identify multivariate biomarkers for quality control of blood plasma biobank specimen. Aliquots of an EDTA plasma pool were kept at 4° C. or −20° C. or −80° C. or in liquid nitrogen, respectively. After 1 day, 5 days, 55 days, 181 days and 365 days, 4 aliquots of samples stored at each temperature were analyzed by metabolite profiling as described in example 4 (sphingolipids were not analyzed in Example 3). Additionally, samples kept at 20° C. were analyzed at t=0 and after 1 day. Plasma samples were analyzed in randomized analytical sequence design. A project pool was generated from aliquots of all samples and measured with 4 replicates within each analytical sequence. The raw peak data was normalized to the median of the project pool per analytical sequence to account for process variability (so called “ratios”). Ratios were log 10 transformed to approach a normal distribution of data.

Samples stored at −80° C. or in liquid nitrogen were considered as high quality samples at any storage time. Additionally, samples analyzed at t=0 or stored at −20° C. for 1 day were considered as high quality samples.

Samples stored at 4° C. were considered as low quality samples at any storage time. Samples stored at −20° C. were considered as low quality samples when stored for 55 days or longer. Other samples were ignored.

### Example 4: Sample Preparation for MS Analysis

Human plasma samples were prepared and subjected to LC-MS/MS and GC-MS or SPE-LC-MS/MS (hormones) analysis as described in the following. Proteins were separated from the blood plasma by precipitation, in particular a neutral buffer was added to the sample and proteins were separated from blood plasma by precipitation, using an appropriate precipitation solvent. After addition of water and a mixture of ethanol and dichloromethane the remaining sample was fractioned into an aqueous, polar phase and an organic, lipophilic phase, in particular by centrifugation.

For the transmethanolysis of the lipid extracts a mixture of 140 μl of chloroform, 37 μl of hydrochloric acid (37% by weight HCl in water), 320 μl of methanol and 20 μl of toluene was added to the evaporated extract. The vessel was sealed tightly and heated for 2 hours at 100° C., with shaking. The solution was subsequently evaporated to dryness. The residue was dried completely.

The methoximation of the carbonyl groups was carried out by reaction with methoxyamine hydrochloride (20 mg/ml in pyridine, 100 l for 1.5 hours at 60° C.) in a tightly sealed vessel. 20 μl of a solution of odd-numbered, straight-chain fatty acids (solution of each 0.3 mg/mL of fatty acids from 7 to 25 carbon atoms and each 0.6 mg/mL of fatty acids with 27, 29 and 31 carbon atoms in 3/7 (v/v) pyridine/toluene) were added as time standards. Finally, the derivatization with 100 μl of N-methyl-N-(trimethylsilyl)-2,2,2-trifluoroacetamide (MSTFA) was carried out for 30 minutes at 60° C., again in the tightly sealed vessel. The final volume before injection into the GC was 220 μl.

For the polar phase the derivatization was performed in the following way: The methoximation of the carbonyl groups was carried out by reaction with methoxyamine hydrochloride (20 mg/ml in pyridine, 50 l for 1.5 hours at 60° C.) in a tightly sealed vessel. 10 μl of a solution of odd-numbered, straight-chain fatty acids (solution of each 0.3 mg/mL of fatty acids from 7 to 25 carbon atoms and each 0.6 mg/mL of fatty acids with 27, 29 and 31 carbon atoms in 317 (v/v) pyridine/toluene) were added as time standards. Finally, the derivatization with 50 μl of N-methyl-N-(trimethylsilyl)-2,2,2-trifluoroacetamide (MSTFA) was carried out for 30 minutes at 60° C., again in the tightly sealed vessel. The final volume before injection into the GC was 110 μl.

The odd-numbered, straight-chain fatty acids included in the above methoximation reactions were included as a time standard for GC, supporting the verification of correct peak annotation.

In particular if small numbers of markers are analyzed, such as those of the panels of the present invention, said time standard is not absolutely necessary.

The GC-MS systems consist of an Agilent 6890 GC coupled to an Agilent 5973 MSD. The autosamplers are CompiPal or GCPal from CTC.

For the analysis usual commercial capillary separation columns (30 m×0.25 mm×0.25 μm) with different poly-methyl-siloxane stationary phases containing 0% up to 35% of aromatic moieties, depending on the analyzed sample materials and fractions from the phase separation step, were used (for example: DB-1 ms, HP-5 ms, DB-XLB, DB-35 ms, Agilent Technologies). Up to 1 μL of the final volume was injected splitless and the oven temperature program was started at 70° C. and ended at 340° C. with different heating rates depending on the sample material and fraction from the phase separation step in order to achieve a sufficient chromatographic separation and number of scans within each analyte peak. Furthermore RTL (Retention Time Locking, Agilent Technologies) was used for the analysis and usual GC-MS standard conditions, for ex-ample constant flow with nominal 1 to 1.7 ml/min. and helium as the mobile phase gas, ionization was done by electron impact with 70 eV, scanning within a m/z range from 15 to 600 with scan rates from 2.5 to 3 scans/sec and standard tune conditions.

The HPLC-MS systems consisted of an Agilent 1100 LC system (Agilent Technologies, Waldbronn, Germany) coupled with an API 4000 Mass spectrometer (Applied Biosystem/MDS SCIEX, Toronto, Canada). HPLC analysis was performed on commercially available reversed phase separation columns with C18 stationary phases (for example: GROM ODS 7 pH, Thermo Betasil C18). Up to 10 μL of the final sample volume of evaporated and reconstituted polar and lipophilic phase was injected and separation was performed with gradient elution using methanol/water/formic acid or acetonitrile/water/formic acid gradients at a flow rate of 200 μL/min.

Mass spectrometry was carried out by electrospray ionization in positive mode for the non-polar fraction and negative or positive mode for the polar fraction using multiple-reaction-monitoring-(MRM)-mode and fullscan from 100-1000 amu.

Analysis of Catecholamines in Plasma Samples:

Catecholamines and their metabolites were measured by online SPE-LC-MS as described by Yamada et al. (Yamada H, Yamahara A, Yasuda S, Abe M, Oguri K, Fukushima S, Ikeda-Wada S: Dansyl chloride derivatization of methamphetamine: a methode with advantages for screening and analysis of methamphetamine in urine. Journal of Analytical Toxicology, 26(1): 17-22 (2002)).

Analysis of Eicosanoids in Plasma Samples

Eicosanoids and related were measured out of plasma by offline- and online-SPE LC-MS/MS (Solid phase extraction-LC-MS/MS) (Masoodi M and Nicolaou A: Rapid Commun Mass Spectrom. 2006; 20(20): 3023-3029. Absolute quantification was performed by means of stable isotope-labelled standards.

Analysis of Sphingoids in Plasma Samples:

In a preferred method, Sphingoids were measured by offline SPE clean-up of the sample and subsequently determined semi-quantitatively by UHPLC-MS/MS: An Oasis® hydrophilic-lipophilic-balanced μElution SPE cartridge (Waters) was conditioned with n-hexane, methanol and methanol/phosphoric acid. After application of the plasma sample, the cartridge was washed with methanol/phosphoric acid before elution of the sphingoids with acetonitrile/isopropanol. The sample was directly injected into the UHPLC-MS/MS system.

Alternatively, metabolites are analyzed in a targeted quantitative mass spectrometry based assay using either a calibration curve or stable isotope labelled internal standards. In this case, the sample preparation (protein precipitation, separation of polar and lipid fractions, and derivatization, if applicable) is done as described above. For detection of the targeted metabolites, the mass spectrometry is carried out in the selected-ion monitoring (SIM) or selected-reaction monitoring (SRM) mode.

### Example 5: Statistical Data Analysis

The software R 2.8.1 (package nlme) was used for data analyses and visualizations. Classification analysis with Random Forest (Liaw and Wiener (2002). Classification and Regression by random Forest. R News 2(3), 18-22.) and Elastic Net (Zou and Hastie (2005) Regularization and variable selection via the elastic net, Journal of the Royal Statistical Society, Series B) was done on log 10 transformed data. The final set of metabolites was determined by considering technological aspects (meaning which biomarker panel can be analyzed together in the sample analytical method setting, e.g. MS based method or enzymatic test based assays); or with respect to the capability to address as many pre-analytical confounders as possible; or with a specific focus on areas of pre-analytics, e.g. processing of blood to plasma or long-term storage; or with a specific focus on matrix check; or with a minimal approach, meaning as few metabolites as possible. For eight metabolites, the intra-sample ratio was calculated meaning that instead or additionally of the ratio versus project pool or versus MxPool™ the quotient of each two metabolites is calculated within each sample and analyzed. This accounts for inter-individual variability.

The resulting classifiers were retrained on the entire merged data of the examples 1-3. We analyzed the low quality samples versus the high quality samples. To analyze the performance of our selected panels, a classifier was built with a random forest or elastic net analysis with these sets of metabolites and the cross validated classification performance was estimated with the area under the curve (AUC) of a receiver operating characteristic (ROC) analysis. Performance calculations were carried out with or without prior ANOVA correction of metabolite data for experiment specific effects on the metabolite baseline levels.

### Example 6: Panel Selection Criteria

Panels of quality markers were selected based on their diagnostic performance to classify high and low quality samples, their quality control objective, their assayability by different analytical methods, their concentration in human plasma, their variability with respect to common variations like fasting, age and gender, and their reproducibility and diagnostic performance in clinical performance validation tests.

### Example 7: Performance of Single Markers

The AUC values obtained with various metabolites as single markers are shown in Table 3. For collection tube related confounders, EDTA plasma was used as the reference as indicated above.

TABLE 3
Univariate AUC values of receiver operating characteristic for the individual metabolites

| Objective | Biomarker (Metabolite(s)) | Direction | AUC |
|---|---|---|---|
| plasma processing | 11-Hydroxyeicosatetraenoic acid | up | 0.5326 |
| related confounders | (C20:cis[5,8,12,14]4) | | |
| blood processing | 12-Hydroxyeicosatetraenoic acid | down | 0.5087 |
| related confounders | (C20:cis[5,8,10,14]4) | | |
| blood processing | 12-Hydroxyheptadecatrienoic acid | down, up | 0.5385 |
| related confounders, | (C17:[5,8,10]3) | | |
| plasma processing | | | |
| related confounders | | | |
| blood processing | 13-Hydroxyoctadecadienoic acid (13-HODE) | up | 0.5053 |
| related confounders | (C18:cis[9]trans[11]2) | | |
| plasma processing | 15-Hydroxyeicosatetraenoic acid | up | 0.5379 |
| related confounders | (C20:cis[5,8,11,13]4) | | |
| plasma processing | 3,4-Dihydroxyphenylacetic acid (DOPAC) | down | 0.7537 |
| related confounders | | | |
| plasma processing | 3,4-Dihydroxyphenylalanine (DOPA) | down | 0.6501 |
| related confounders | | | |
| plasma processing | 3,4-Dihydroxyphenylglycol (DOPEG) | down | 0.8895 |
| related confounders | | | |
| plasma processing | 3-Phosphoglycerate (3-PGA) | up | 0.71 |
| related confounders | | | |
| plasma processing | 5-Hydroxyeicosatetraenoic acid | up | 0.5322 |
| related confounders | (C20:trans[6]cis[8,11,14]4) (5-HETE) | | |
| plasma processing | 8,9-Dihydroxyeicosatrienoic acid | up | 0.5349 |
| related confounders | (C20:cis[5,11,14]3) | | |
| plasma processing | 8-Hydroxyeicosatetraenoic acid | up | 0.5751 |
| related confounders | (C20:trans[5]cis[9,11,14]4) (8-HETE) | | |
| plasma processing | 9-Hydroxyoctadecadienoic acid (9-HODE) | up | 0.5415 |
| related confounders | (C18:trans[10]cis[12]2) | | |
| plasma processing | Adrenaline (Epinephrine) | down | 0.7327 |
| related confounders | | | |
| plasma processing | Alanine | up | 0.5897 |
| related confounders | | | |
| blood processing | Arginine | down | 0.5951 |
| related confounders | | | |
| blood processing | Arginine | down | 0.5747 |
| related confounders | | | |
| plasma processing | Asparagine | down | 0.506 |
| related confounders | | | |
| collection tube | Aspartate | up | 0.5164 |
| plasma processing | Aspartate/Asparagine intra-sample ratio | up | 0.5926 |
| related confounders | | | |
| plasma processing | Ceramide (d18:1,C24:0) | up | 0.5414 |
| related confounders | | | |
| plasma processing | Cholesterylester hydroperoxide (C18:2-9-OOH), | up | 0.7213 |
| related confounders | Cholesterylester hydroperoxide (C20:4-OOH), | | |
| | Cholesterylester hydroperoxide (C18:2-13- | | |
| | OOH) | | |
| collection tube | Citrate | up | 0.681 |
| blood processing | Citrulline | up | 0.5201 |
| related confounders | | | |
| blood processing | Creatinine | up | 0.5785 |
| related confounders | | | |
| plasma processing | Cysteine | down | 0.7206 |
| related confounders | | | |
| plasma processing | Cystine | down | 0.7023 |
| related confounders | | | |
| collection tube | Ethylenediaminetetraacetic acid (EDTA) | down | na |
| blood processing | Glucose | down | 0.7584 |
| related confounders | | | |
| blood processing | Glucose-6-phosphate | up | 0.697 |
| related confounders | | | |
| plasma processing | Glutamate | up | 0.5399 |
| related confounders | | | |
| plasma processing | Glutamate/Glutamine intra-sample ratio | up | 0.6667 |
| related confounders | | | |
| plasma processing | Glutamine | down | 0.7344 |
| related confounders | | | |
| plasma processing | Glycerate | up | 0.5541 |
| related confounders | | | |
| plasma processing | Glycerol-3-phosphate | up | 0.5843 |
| related confounders | | | |
| blood processing | Hypoxanthine | up | 0.6212 |
| related confounders | | | |
| blood processing | Lactate | up | 0.517 |
| related confounders | | | |
| blood processing | Lactate/Glucose intra-sample ratio | up | 0.5484 |
| related confounders | | | |
| plasma processing | Lysophosphatidylcholine (C17:0) | up | 0.5873 |
| related confounders | | | |
| plasma processing | Lysophosphatidylcholine (C18:0) | up | 0.5207 |
| related confounders | | | |
| plasma processing | Lysophosphatidylcholine (C18:1) | up | 0.555 |
| related confounders | | | |
| plasma processing | Lysophosphatidylcholine (C20:4) | up | 0.5008 |
| related confounders | | | |
| blood processing | Maltose | up | 0.6564 |
| related confounders | | | |
| blood processing | Maltotriose | up | 0.8762 |
| related confounders | | | |
| plasma processing | Noradrenaline (Norepinephrine) | down | 0.9047 |
| related confounders | | | |
| blood processing | Ornithine | up | 0.5837 |
| related confounders | | | |
| blood processing | Ornithine/Arginine intra-sample ratio | up | 0.5165 |
| related confounders | | | |
| blood processing | Pentoses | up | 0.5349 |
| related confounders | | | |
| plasma processing | Phosphatidylcholine hydroperoxide | up | 0.695 |
| related confounders | (C16:0,C18:1-OOH) | | |
| plasma processing | Phosphatidylcholine hydroperoxide | up | 0.614 |
| related confounders | (C16:0,C18:2-OOH) | | |
| plasma processing | Phosphatidylcholine hydroperoxide | up | 0.5574 |
| related confounders | (C18:0,C18:2-OOH) | | |
| plasma processing | Prostaglandin D2 | up | 0.5875 |
| related confounders | | | |
| plasma processing | Prostaglandin E2 | up | 0.5868 |
| related confounders | | | |
| blood processing | Ribose | up | 0.5123 |
| related confounders | | | |
| blood processing | Serotonin (5-HT) | down | 0.6164 |
| related confounders | | | |
| blood processing | Sphingadienine (d18:2) | down | 0.6685 |
| related confounders | | | |
| blood processing | Sphingadienine-1-phosphate (d18:2) | up | 0.5997 |
| related confounders | | | |
| blood processing | Sphingosine (d16:1) | down | 0.8343 |
| related confounders | | | |
| blood processing | Sphingosine (d18:1) | down | 0.8904 |
| related confounders | | | |
| blood processing | Sphingosine-1-phosphate (d16:1) | up | 0.5284 |
| related confounders | | | |
| blood processing | Sphingosine-1-phosphate (d17:1) | up | 0.6247 |
| related confounders | | | |
| blood processing | Sphingosine-1-phosphate (d18:1) | up | 0.798 |
| related confounders | | | |
| blood processing | Taurine | down | 0.6343 |
| related confounders | | | |
| plasma processing | Threonic acid | up | 0.5448 |
| related confounders | | | |
| blood processing | Thromboxane B2 | down, up | 0.6766 |
| related confounders, | | | |
| plasma processing | | | |
| related confounders | | | |
| plasma processing | Triacylgyceride hydroperoxide | up | 0.6769 |
| related confounders | (C16:0,C18:1,C18:2-OOH) | | |
| plasma processing | Triacylgyceride hydroperoxide | up | 0.6584 |
| related confounders | (C16:0,C18:1,C18:3-OOH), Triacylgyceride | | |
| | hydroperoxide (C16:0,C18:2,C18:2-OOH) | | |
| plasma processing | Triacylgyceride hydroperoxide | up | 0.5225 |
| related confounders | (C16:0,C18:1,C20:4-OOH), Triacylgyceride | | |
| | hydroperoxide (C18:1,18:2,C18:2-OOH), | | |
| | Triacylgyceride hydroperoxide | | |
| | (C18:1,C18:1,C18:3-OOH) | | |
| blood processing | Uric acid | up | 0.655 |
| related confounders | | | |

### Example 8: Performance of Optimized Panels

Based on the criterion optimal performance and further criteria as noted above (Examples 5 and 6), panels were selected as specified above, resulting in panels summarized in Table 2. Performances of said panels of Table 2, expressed as AUC estimates, are shown in Table 4.

TABLE 4
Performance (estimated AUC values) of metabolite/marker panels suited
for quality control of plasma samples detecting pre-analytical confounders.

| | | Elastic Net, | Random | Random |
|---|---|---|---|---|
| Panel | Elastic Net, | incl. | Forest, | Forest, |
| Number | excl. ANOVA | ANOVA | excl. ANOVA | incl. ANOVA |
| 1 | 0.90 | 0.94 | 0.99 | 0.99 |
| 2 | 0.94 | 0.98 | 0.98 | 0.99 |
| 3 | 0.90 | 0.97 | 0.99 | 1.00 |
| 4 | 0.77 | 0.86 | 0.79 | 0.86 |
| 5 | 0.74 | 0.78 | 0.84 | 0.89 |
| 6 | 0.71 | 0.85 | 0.96 | 0.97 |
| 7 | 0.73 | 0.50 | 0.78 | 0.77 |
| 8 | 0.86 | 0.96 | 0.94 | 0.99 |
| 9 | 0.69 | 0.67 | 0.79 | 0.75 |
| 10 | 0.91 | 0.97 | 0.99 | 0.99 |
| 11 | 0.89 | 0.97 | 0.98 | 0.99 |
| 12 | 0.79 | 0.86 | 0.95 | 0.95 |
| 13 | 0.88 | 0.96 | 0.96 | 0.98 |
| 14 | 0.88 | 0.96 | 0.98 | 0.99 |
| 15 | 0.90 | 0.96 | 0.98 | 0.99 |
| 16 | 0.89 | 0.97 | 0.99 | 1.00 |
| 17 | 0.92 | 0.99 | 0.98 | 0.99 |
| 18 | 0.85 | 0.95 | 0,96 | 0.99 |
| 19 | 0.75 | 0.95 | 0.95 | 0.98 |
| 20 | 0.85 | 0.95 | 0.95 | 0.98 |

TABLE 4a
Performance (estimated AUC values) of metabolite/marker panels suited
for quality control of plasma samples detecting pre-analytical confounders
with additional inclusion of “matrix-check markers” (Panel 9)

| | | Elastic Net, | Random | Random |
|---|---|---|---|---|
| Panel | Elastic Net, | incl. | Forest, | Forest, |
| Number | excl. ANOVA | ANOVA | excl. ANOVA | incl. ANOVA |
| 3_m | 0.90 | 0.97 | 0.99 | 0.99 |
| 6_m | 0.77 | 0.85 | 0.97 | 0.96 |
| 10_m | 0.91 | 0.97 | 0.99 | 1.00 |
| 15_m | 0.90 | 0.97 | 0.98 | 0.99 |
| 16_m | 0.89 | 0.97 | 0.99 | 1.00 |
| 17_m | 0.92 | 0.99 | 0.99 | 1.00 |
| 18_m | 0.88 | 0.95 | 0.96 | 0.99 |
| 19_m | 0.83 | 0.95 | 0.97 | 0.99 |
| 20_m | 0.87 | 0.95 | 0.97 | 0.99 |

### Example 9: Performance of Sub-Panels

Optimized panels of Table 2 were examined for markers occurring frequently. Specific combinations of such frequently identified markers could be combined into sub-panels (Table 1) having surprisingly high performance (Table 5).

TABLE 5
Performance (estimated AUC values) of minimal marker panels suited
for quality control of plasma samples detecting pre-analytical confounders.

| | AUC estimate | | | |
|---|---|---|---|---|
| | | Elastic Net, | Random | Random |
| Panel | Elastic Net, | incl. | Forest, | Forest, |
| Number | excl. ANOVA | ANOVA | excl. ANOVA | incl. ANOVA |
| 1_a | 0.65 | 0.62 | 0.76 | 0.77 |
| 1_b | 0.65 | 0.66 | 0.79 | 0.79 |
| 10_a | 0.86 | 0.95 | 0.93 | 0.97 |
| 11_a | 0.76 | 0.94 | 0.94 | 0.97 |
| 12_a | 0.76 | 0.75 | 0.85 | 0.85 |
| 13_a | 0.74 | 0.95 | 0.89 | 0.98 |
| 13_b | 0.72 | 0.86 | 0.95 | 0.96 |
| 14_a | 0.66 | 0.95 | 0.85 | 0.95 |
| 14_b | 0.79 | 0.88 | 0.89 | 0.92 |
| 15_a | 0.79 | 0.75 | 0.82 | 0.83 |
| 16_a | 0.85 | 0.95 | 0.95 | 0.98 |
| 17_b | 0.89 | 0.89 | 0.96 | 0.96 |
| 18_b | 0.74 | 0.79 | 0.91 | 0.95 |
| 19_a | 0.74 | 0.95 | 0.90 | 0.97 |
| 19_b | 0.67 | 0.94 | 0.89 | 0.95 |
| 2_b | 0.72 | 0.86 | 0.95 | 0.95 |
| 20_b | 0.66 | 0.82 | 0.88 | 0.93 |
| 3_a | 0.83 | 0.95 | 0.89 | 0.97 |
| 3_b | 0.78 | 0.79 | 0.90 | 0.89 |
| 4_a | 0.60 | 0.81 | 0.78 | 0.89 |
| 5_a | 0.72 | 0.74 | 0.79 | 0.83 |
| 5_b | 0.72 | 0.76 | 0.80 | 0.84 |
| 6_a | 0.69 | 0.78 | 0.89 | 0.91 |
| 7_a | 0.65 | 0.50 | 0.77 | 0.76 |
| 7_b | 0.70 | 0.50 | 0.78 | 0.78 |
| 8_b | 0.67 | 0.95 | 0.87 | 0.92 |

### Example 10: Experimental Design of Creating Samples of High Quality and Low Quality with Respect to Processing of Blood to Serum

In order to explicitly show that the panels identified for quality control of plasma are also applicable to serum, blood was taken from 20 healthy volunteers.

Different groups of sample handling comprised the following procedures:
Prolonged clotting of blood
Prolonged incubation of serum at room temperature

Twenty healthy volunteers (15 females, 5 males) were recruited and blood was withdrawn by venous puncture using a gauge-20 safety-fly blood collection system into 2 blood collection tubes without anticoagulant. The blood of each subject was processed within the different groups as follows:

Control

For each subject, one of the blood collection tubes was incubated for 40 min at room temperature and the serum was prepared by centrifugation at 2000×g for 20 minutes in a temperature-controlled centrifuge at 20° C. The supernatant serum was gently mixed in a fresh tube and stored in aliquots at −80° C. until analysis.

Prolonged Clotting of Blood

For each subject, one of the blood collection tubes was incubated for 6 h at room temperature and the serum was prepared by centrifugation at 2000×g for 20 minutes in a temperature-controlled centrifuge at 20° C. The supernatant serum was gently mixed in a fresh tube and stored in aliquots at −80° C. until analysis.

Prolonged Incubation of Serum at Room Temperature

Aliquots of the control group sera were incubated at room temperature for 24 h before freezing and storage at −80° C. until analysis.

The serum samples of this experiment were analyzed by MxP® Broad Profiling as described in example 4 in randomized analytical sequence design and following the pool and MxPool™ concept as described in example 2. Samples of the control group are considered to be of high quality, the other samples from this experiment are considered to be of low quality. Selected panels were analyzed for their performances to identify low quality samples and distinguish them from the control samples as described in example 5 using elastic net algorithm. The panel numbers refer to the metabolite lists given by Tables 1-2. AUC estimates of the panels are shown in Tables 6-7.

TABLE 6
Performance (estimated AUC values) of metabolite/marker panels
especially suited for quality control of serum samples
by detecting pre-analytical confounders.

| | AUC estimate, Elastic Net, |
|---|---|
| Panel Number | excl. ANOVA |
| 3 | 0.99990 |
| 15 | 0.99948 |
| 16 | 0.99995 |
| 18 | 0.99495 |
| 19 | 0.99503 |
| 20 | 0.99686 |

TABLE 7
Performance (estimated AUC values) of minimal marker panels
especially suited for quality control of serum samples
by detecting pre-analytical confounders.

| | AUC estimate, Elastic Net, |
|---|---|
| Panel Number | excl. ANOVA |
| 1_a | 0.97566 |
| 2_b | 0.99035 |
| 3_a | 0.97746 |
| 3_b | 0.98802 |
| 6_a | 0.98185 |
| 8_b | 0.97303 |
| 10_a | 0.99218 |
| 11_a | 0.99368 |
| 13_a | 0.99393 |
| 13_b | 0.99133 |
| 14_a | 0.99795 |
| 15_a | 0.97722 |
| 16_a | 0.97866 |
| 18_b | 0.97386 |
| 19_a | 0.99364 |
| 19_b | 0.99611 |
| 20_b | 0.99900 |

### Example 11: Experimental Design to Show Application of Quality Control of Samples with Respect to Other Down-Stream Analysis

In order to explicitly show that quality control as described in this invention is also applicable to other down-stream applications such as protein analysis and allows for estimation of suitability of biobank samples or clinical trial samples for other applications, blood was taken from healthy volunteers and processed to plasma.

Different groups of sample handling comprised the following procedures:
Prolonged incubation of EDTA blood previous to centrifugation
Prolonged incubation of plasma at room temperature

Twenty healthy volunteers (15 females, 5 males) were recruited and blood was withdrawn by venous puncture using a gauge-20 safety-fly blood collection system into three K3EDTA blood collection tubes. The blood of each subject was processed within the different groups as follows:

Control

For each subject, one of the blood collection tubes was processed without any delay and the plasma was prepared by centrifugation at 2500×g for 10 minutes in a temperature-controlled centrifuge at 20° C. The supernatant plasma was transferred to another centrifugation tube and centrifuged again at 16000×g for 10 min in a temperature-controlled centrifuge at 20° C. The supernatant plasma was gently mixed in a fresh tube and stored in aliquots at −80° C. until analysis.

Prolonged Incubation of Blood

For each subject, one of the blood collection tubes was incubated for 6 h at room temperature and subsequently the plasma was prepared by centrifugation at 2500×g for 10 minutes in a temperature-controlled centrifuge at 20° C. The supernatant plasma was transferred to another centrifugation tube and centrifuged again at 16000×g for 10 min in a temperature-controlled centrifuge at 20° C. The supernatant plasma was gently mixed in fresh tube and stored in aliquots at −80° C. until analysis.

Prolonged Incubation of Plasma

For each subject, one of the blood collection tubes was processed without any delay and the plasma was prepared by centrifugation at 2500×g for 10 minutes in a temperature-controlled centrifuge at 20° C. The supernatant plasma was transferred to another centrifugation tube and centrifuged again at 16000×g for 10 min in a temperature-controlled centrifuge at 20° C. The supernatant plasma was gently mixed in a fresh tube and incubated for 24 h at room temperature before freezing and stored in aliquots at −80° C. until analysis.

Proteins were analyzed by methods well known to those skilled in the art and that are applied in routine clinical chemistry laboratories. Those methods comprise Enzyme Linked Immunosorbent Assay (ELISA), Radio Immuno Assay (RIA), Electro-chemiluminescence binding assay (ECLIA), or other assays.

Statistical analysis was done using a paired t-test of log 10 transformed protein concentrations. Each protein was tested for its significant difference in the blood processing related confounded group or the plasma processing related confounded group relative to the control group. Results are shown in tables 8-9.

TABLE 8
Susceptibility of proteins in plasma to blood processing related pre-analytical variation

| | | Blood stored for 6 h previous to | | | |
|---|---|---|---|---|---|
| | | centrifugation relative to control | | | |
| | | at room temperature | | | |
| | | | | p-value | |
| | | | | of | t-value of |
| | | Mean | Mean | paired | paired t- |
| Protein | Unit | difference | ratio | t-test | test |
| Adrenocorticotropic hormone | ng/l | −0.090 | 0.988 | 0.2117 | −1.2925 |
| (ACTH) | | | | | |
| Antidiuretic hormone (ADH; | ng/l | 0.635 | 1.421 | 0.0063 | 3.0675 |
| vasopressin) | | | | | |
| Angiotensin II | ng/l | −2.190 | 0.739 | 0.7688 | 0.3030 |
| alpha-2-HS-glycoprotein (AHSG; | g/l | 0.042 | 0.995 | 0.5660 | 0.5847 |
| fetuin-A) | | | | | |
| Human FGF-23 c-terminal | kRU/l | −1.750 | 0.996 | 0.8488 | −0.1935 |
| Fibronectin | g/l | −0.015 | 0.959 | 0.3407 | −0.9772 |
| Glucagon | ng/l | −1.100 | 0.989 | 0.1951 | −1.3607 |
| Insulin | μU/ml | 0.105 | 1.025 | 0.2168 | 1.2777 |
| Prolactin | ng/ml | 0.058 | 1.009 | 0.1441 | 1.5234 |
| Parathyroid hormone (PTH) | pmol/l | 0.027 | 1.006 | 0.5787 | 0.5650 |
| Renin | pg/ml | −0.026 | 1.009 | 0.4337 | 0.7999 |
| Thyroid-stimulating hormone (TSH) | mU/l | 0.004 | 1.004 | 0.3896 | 0.8805 |

TABLE 9
Susceptibility of proteins in plasma to plasma processing related pre-analytical variation

| | | Plasma stored for 24 h relative to control at | | | |
|---|---|---|---|---|---|
| | | room temperature | | | |
| | | | | | t-value of |
| | | Mean | Mean | p-value of | paired t- |
| Protein | Unit | difference | ratio | paired t-test | test |
| Adrenocorticotropic hormone | ng/l | −1.365 | 0.932 | 5.9357E−09 | −9.9255 |
| (ACTH) | | | | | |
| Antidiuretic hormone (ADH; | ng/l | 0.455 | 1.308 | 0.0095 | 2.8841 |
| vasopressin) | | | | | |
| Angiotensin II | ng/l | 6.515 | 1.079 | 0.0009 | 4.8809 |
| alpha-2-HS-glycoprotein (AHSG; | g/l | 0.058 | 1.024 | 0.0299 | 2.3585 |
| fetuin-A) | | | | | |
| Human FGF-23 c-terminal | kRU/l | −4.900 | 0.916 | 0.3958 | −0.8741 |
| Fibronectin | g/l | −0.020 | 0.920 | 0.1011 | −1.7229 |
| Glucagon | ng/l | 10.450 | 1.307 | 0.4426 | −0.7902 |
| Insulin | μU/ml | −0.070 | 0.991 | 0.0789 | −1.8566 |
| Prolactin | ng/ml | 0.020 | 1.002 | 0.6555 | 0.4532 |
| Parathyroid hormone (PTH) | pmol/l | 0.087 | 1.027 | 0.0601 | 1.9989 |
| Renin | pg/ml | −1.188 | 0.938 | 5.7391E−06 | −6.2122 |
| Thyroid-stimulating hormone (TSH) | mU/l | 0.004 | 1.004 | 0.0958 | 1.7527 |

The data of Tables 8 and 9 show that samples identified as samples of low quality according to the method of the present invention show significant changes in the activities and/or concentration of proteins tested and are, accordingly, of insufficient quality for, e.g., diagnostic or proteomic purposes.

1. A method for assessing the quality of a blood product sample, comprising:
a) determining in said sample the values of the markers of at least one panel of Table 1;
b) comparing the values determined in step a) with corresponding references, and,
c) assessing the quality of said blood product sample.

2. The method of claim 1, wherein in step a) the amounts of the markers (i) glycerol-3-phosphate, glycerate, and ornithine; (ii) glycerol-3-phosphate, glycerate, and hypoxanthine; (iii) glycerol-3-phosphate, ornithine, and hypoxanthine; or (iv) glycerate, ornithine, and hypoxanthine are determined.

3. The method of claim 1, wherein in step a) the values of the markers of at least one panel of Table 2 are determined.

4. The method of claim 1, wherein in step a) the values of the markers of at least one of panels 3, 13, 15, 18, 19, or 20 of Table 2 are determined.

5. The method of claim 1, wherein said determining the value of a marker is determining the amount of said marker or is determining a calculated value derived from at least one concentration value of a marker, preferably, a ratio of the concentrations of at least two biomarkers.

6. The method of claim 1, wherein the individual numerical values of said markers are translated into a combined value by using a multivariate model, preferably, a logistic regression model.

7. The method of claim 1, wherein said step b) comprises the steps of
b1) calculating a combined value based on the determined values of said markers as referred to in step a), wherein, preferably, in said calculating a combined value the markers are weighted due to their importance; and
b2) comparing the, thus, calculated combined value to a reference combined value.

8. The method of claim 1, wherein said step b) comprises the steps of
b1) comparing the values determined in step a) with corresponding references, and calculating a combined value based on said comparison, wherein, preferably, in said calculating a combined value the markers are weighted due to their importance; and
b2) comparing the, thus, calculated combined value to a reference combined value.

9. The method of claim 1, wherein assessing the quality of a blood product sample is ensuring that said blood product sample has not been affected by any of the confounding factors (i) prolonged time between phlebotomy and separation of plasma from blood cells, (ii) increased temperature between phlebotomy and separation of plasma from blood cells, (iii) prolonged time of storage of plasma, and (iv) increased temperature during storage of plasma.

10. The method of claim 1, wherein in step a) additionally the amounts of the markers ethylenediaminetetraacetic acid (EDTA), citrate, and aspartate are determined, and wherein in step b) the amounts of said additional markers are compared to corresponding references.

11. The method of claim 10, wherein assessing the quality of a blood product sample further comprises differentiating whether said sample has been compromised by factors related to collection tube selection.

12. The method of claim 1, wherein said blood product sample is a blood sample or a plasma sample.

13. A device for assessing the quality of a blood product sample comprising:
a) an analyzing unit for said sample, comprising at least one detector for at least the markers of at least one panel of Table 1, Table 2, or Table 2a, said at least one detector determining the amounts of said markers in said sample; and, operatively linked thereto,
b) an evaluation unit comprising a data processing unit and a database, said data base comprising stored corresponding reference values and said data processing unit optionally having tangibly embedded an algorithm calculating an intra-sample ratio of two biomarkers and comparing the values of the markers determined by the analyzing unit or the values calculated by the evaluation unit to said stored reference values and generating an output information based on which assessment of the quality is established.

14. The device of claim 13, wherein said analyzing unit comprises at least one detector for at least the markers glycerol-3-phosphate, glycerate, and ornithine, said at least one detector determining the amounts of said markers in said sample.

15. A data collection comprising characteristic values of at least the markers of at least one panel of Table 1, being indicative for sufficient or insufficient quality of a blood product sample.

16. A data storage medium comprising the data collection of claim 15.

17. Use of at least the markers of at least one panel of Table 1, or of a detection agent or detection reagents therefor, for assessing the quality of a blood product sample.

18. A kit for assessing the quality of a blood product sample comprising at least one detection agent for at least the markers of at least one panel of Table 1, and/or references for the said markers, comprised in a housing.

19. A method of providing a collection of blood products of sufficient quality, comprising
a) providing a collection of blood products,
b) performing the steps of the method for assessing the quality of a blood product sample of any one of claim 1 on a sample of each member of said collection of blood products,
c) discarding a blood product in case insufficient quality is assessed, and/or excluding a blood product from further use in case insufficient quality is assessed; thereby providing a collection of blood products of sufficient quality.
